SEQUENCE LISTING (33 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 29 aa, synthetic construct
  REGION 1..29: note = B chain
FVRQHLCGSHLVEALELVCGERGFHYTPK

SEQ ID NO: 4 - protein, 29 aa, synthetic construct
  REGION 1..29: note = B chain
FVRQHLCGSHLVEALELVCGERGFHYRPK

SEQ ID NO: 5 - protein, 29 aa, synthetic construct
  REGION 1..29: note = B chain
FVNQHLCGSHLVEALELVCGERGFHYRPK

SEQ ID NO: 6 - protein, 29 aa, synthetic construct
  REGION 1..29: note = B chain
FVNQHLCGSHLVEALELVCGERGFHYTPK

SEQ ID NO: 7 - protein, 29 aa, synthetic construct
  REGION 1..29: note = B chain
FVNQHLCGSHLVEALHLVCGERGFHYTPK

SEQ ID NO: 8 - protein, 31 aa, synthetic construct
  REGION 1..31: note = B chain
FVNQHLCGSHLVEALELVCGERGFHYTPKTR

SEQ ID NO: 9 - protein, 31 aa, synthetic construct
  REGION 1..31: note = B chain
FVNQHLCGSHLVEALELVCGERGFHYRPKTR

SEQ ID NO: 10 - protein, 31 aa, synthetic construct
  REGION 1..31: note = B chain
FVNQHLCGSHLVEALHLVCGERGFHYRPKTR

SEQ ID NO: 11 - protein, 21 aa, synthetic construct
  REGION 1..21: note = A chain
GIVERCCTSICSLERLERYCG

SEQ ID NO: 12 - protein, 21 aa, synthetic construct
  REGION 1..21: note = A chain
GIVEQCCTSICSLERLERYCR

SEQ ID NO: 13 - protein, 21 aa, synthetic construct
  REGION 1..21: note = A chain
GIVERCCTSICSLEQLERYCG

SEQ ID NO: 14 - protein, 21 aa, synthetic construct
  REGION 1..21: note = A chain
GIVERCCTSICSLEQLERYCR

SEQ ID NO: 15 - protein, 21 aa, synthetic construct
  REGION 1..21: note = A chain
GIVERCCTSICSLERLENYCR

SEQ ID NO: 16 - protein, 21 aa, synthetic construct
  REGION 1..21: note = A chain
GIVERCCTSICSLERLERYCR

SEQ ID NO: 17 - protein, 21 aa, synthetic construct
  REGION 1..21: note = A chain
GIVERCCTRICSLERLERYCR

SEQ ID NO: 18 - protein, 21 aa, synthetic construct
  REGION 1..21: note = A chain
GIVERCCTRICSLERLERYCG

SEQ ID NO: 19 - protein, 21 aa, synthetic construct
  REGION 1..21: note = A chain
GIVEQCCTRICSLERLERYCR

SEQ ID NO: 20 - protein, 21 aa, synthetic construct
  REGION 1..21: note = A chain
GIVERCCTRICSLEQLERYCR

SEQ ID NO: 21 - protein, 21 aa, synthetic construct
  REGION 1..21: note = A chain
GIVERCCTRICSLERLENYCR

SEQ ID NO: 22 - protein, 21 aa, synthetic construct
  REGION 1..21: note = A chain
GIVERCCTSICSLDRLERYCG

SEQ ID NO: 23 - protein, 21 aa, synthetic construct
  REGION 1..21: note = A chain
GIVERCCTSICSLDRLERYCG

SEQ ID NO: 24 - protein, 21 aa, synthetic construct
  REGION 1..21: note = A chain
GIVERCCTSICSLDQLERYCG

SEQ ID NO: 25 - protein, 21 aa, synthetic construct
  REGION 1..21: note = A chain
GIVEQCCTSICSLDRLERYCR

SEQ ID NO: 26 - protein, 21 aa, synthetic construct
  REGION 1..21: note = A chain
GIVERCCTSICSLDQLERYCR

SEQ ID NO: 27 - protein, 21 aa, synthetic construct
  REGION 1..21: note = A chain
GIVERCCTSICSLDRLENYCR

SEQ ID NO: 28 - protein, 21 aa, synthetic construct
  REGION 1..21: note = A chain
GIVERCCTSICSLDRLERYCR

SEQ ID NO: 29 - protein, 21 aa, synthetic construct
  REGION 1..21: note = A chain
GIVERCCTRICSLDRLERYCR

SEQ ID NO: 30 - protein, 21 aa, synthetic construct
  REGION 1..21: note = A chain
GIVERCCTRICSLDRLERYCG

SEQ ID NO: 31 - protein, 21 aa, synthetic construct
  REGION 1..21: note = A chain
GIVEQCCTRICSLDRLERYCR

SEQ ID NO: 32 - protein, 21 aa, synthetic construct
  REGION 1..21: note = A chain
GIVERCCTRICSLDQLERYCR

SEQ ID NO: 33 - protein, 21 aa, synthetic construct
  REGION 1..21: note = A chain
GIVERCCTRICSLDRLENYCR

(19) United States
(12) Patent Application Publication
(10) Pub. No.: US 2025/0082758 A1
(43) Pub. Date: Mar. 13, 2025

(54) INSULIN CONJUGATES

(71) Applicant: SANOFI, Paris (FR)

(72) Inventor: Thomas Boehme, Frankfurt am Main (DE)

(21) Appl. No.: 18/730,532

(22) PCT Filed: Jan. 26, 2023
(86) PCT No.: PCT/EP2023/051946

§ 371 Date: Jul. 19, 2024

(30) Foreign Application Priority Data
Jan. 26, 2022 (EP) 22315022.8

(51) Int. Cl. A61K 47/54; A61K 38/28

(52) U.S. Cl. CPC A61K 47/543; A61K 38/28

(57) ABSTRACT

The present invention relates to an insulin conjugate comprising a human serum albumin binder of Formula (I) and a human insulin analog, wherein the human serum albumin binder of Formula (I) is covalently bound to the human insulin analog in that the terminal carboxy group “a” of the human serum albumin binder of Formula (I) is covalently bound via an amide bond to the epsilon amino group of lysine B29 of the human insulin analog. As well as related aspects such as a pharmaceutical composition, use in medicine, methods for preparing such insulin conjugates and so forth.

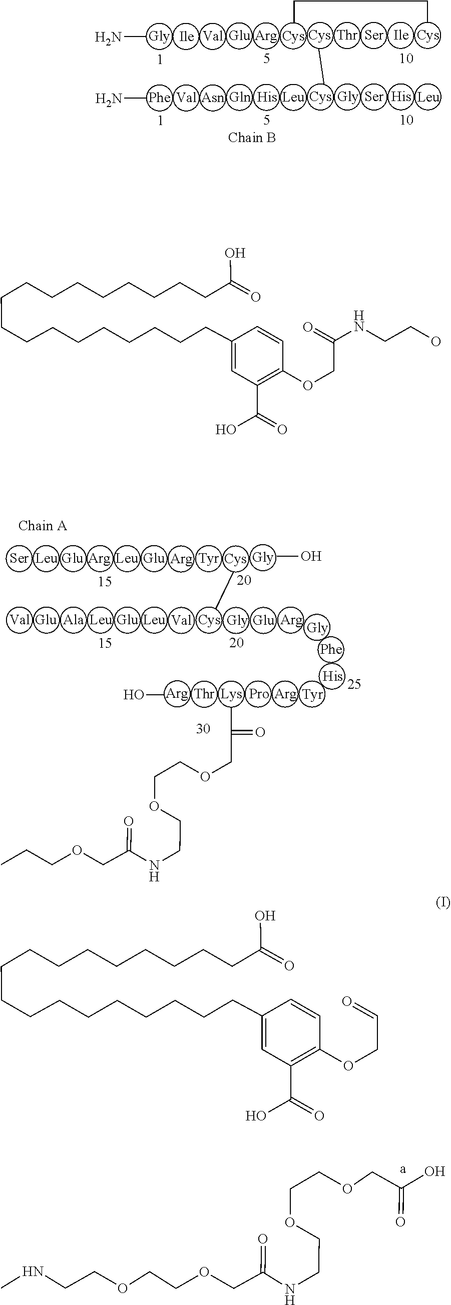

### FIELD

The present invention relates to an insulin conjugate comprising a human serum albumin binder of Formula (I) and a human insulin analog, wherein the human serum albumin binder of Formula (I) is covalently bound to the human insulin analog in that the terminal carboxy group “a” of the human serum albumin binder of Formula (I) is covalently bound via an amide bond to the epsilon amino group of lysine B29 of the human insulin analog. As well as related aspects such as a pharmaceutical composition, use in medicine, methods for preparing such insulin conjugates and so forth.

### BACKGROUND

Worldwide, more than 400 million people suffer from type 1 or type 2 diabetes mellitus. Type 1 diabetes is treated with insulin substitution. In contrast to type 1 diabetes, there is basically no deficiency of insulin in type 2 diabetes, but in a large number of cases, especially in the advanced stage, type 2 diabetes patients are treated with insulin.

In a healthy person, the release of insulin by the pancreas is strictly coupled to the concentration of the blood glucose. Elevated blood glucose levels, occur after meals, and are rapidly compensated by a corresponding increase in insulin secretion. In the fasting state, the plasma insulin level falls to a basal value which is adequate to guarantee a continuous supply of insulin-sensitive organs and tissue with glucose and to keep hepatic glucose production low in the night. Often, the replacement of the endogenous insulin secretion by exogenous, mostly subcutaneous administration of insulin does not achieve the quality of the physiological regulation of the blood glucose described above. Deviations of the blood glucose upward or downward can occur, which in their severest forms can be life-threatening. It is to be derived from this that an improved therapy of diabetes is primarily to be aimed at keeping the blood glucose as closely as possible in the physiological range.

Human insulin is a polypeptide of 51 amino acids, which are divided into 2 amino acid chains: the A chain having 21 amino acids and the B chain having 30 amino acids. The chains are connected to one another by means of two disulfide bridges. A third disulfide bridge exists between the cysteines at position 6 and 11 of the A chain. Some products in current use for the treatment of diabetes mellitus contain are insulin conjugates, i.e. insulin variants whose sequence differs from that of human insulin by one or more amino acid substitutions in the A chain and/or in the B chain.

Like many other peptide hormones, human insulin has a short half-life in vivo. Thus, it is administered frequently which is associated with discomfort for the patient. Therefore, insulin conjugates are desired which have an increased half-life in vivo and, thus, a prolonged duration of action.

There are currently different approaches for extending the half-life of insulins.

One approach is based on the development of a soluble formulation at low pH, but of reduced solubility relative to native insulin at physiologic pH. The isoelectric point of the insulin conjugate is increased through the addition of two arginines to the C-terminus of the B-chain. The addition of two arginines in combination with a glycine substitution at A21 (insulin glargine) provides an insulin with extended duration of action. The insulin conjugate precipitates in the presence of zinc upon injection in subcutaneous sites and slowly solubilizes, resulting a sustained presence of insulin glargine.

WO2016006963 A1 (Jung, et al., HANMI PHARM. CO., LTD.) discloses insulin conjugates having a reduced insulin receptor-mediated clearance rate, compared to human insulin.

WO2018056764 A1 (Choi, et al., HANMI PHARM. & Sanofi) discloses insulin conjugates having a reduced insulin receptor-mediated clearance rate, compared to human insulin.

WO2008034881 A1 (Nielsen, et al., NOVO NORDISK A/S) discloses protease stabilized insulin conjugates.

In another approach, a long chain fatty acid group is conjugated to the epsilon amino group of LysB29 of insulin. The presence of this group allows the attachment of the insulin to serum albumin by non-covalent, reversible binding. As a consequence, this insulin conjugate has a significantly prolonged time-action profile relative to human insulin (see e.g. Mayer et al., Inc. Biopolymers [Pept Sci]88: 687-713, 2007; or WO2009115469 A1 [Madsen, et al., NOVO NORDISK A/S]).

None of the prior art teach or provide solutions that fulfil all the patients' needs in the present context.

### SUMMARY

A first aspect of the invention relates to an insulin conjugate comprising a human serum albumin binder of Formula (I) and a human insulin analog, wherein the human serum albumin binder of Formula (I) is covalently bound to the human insulin analog in that the terminal carboxy group “a” of the human serum albumin binder of Formula (I) is covalently bound via an amide bond to the epsilon amino group of lysine B29 of the human insulin analog.

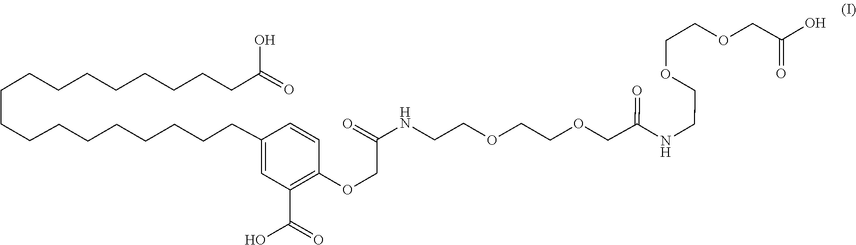 [SMILES: O=C(O)CCCCCCCCCCCCCCCCCCc1ccc(OCC(=O)NCCOCCOCC(=O)NCCOCCOCC(=O)O)c(C(=O)O)c1]

The first aspect also relates to certain human insulin analogs.

A second aspect of the invention relates to a pharmaceutical composition comprising in a pharmaceutically effective amount the insulin conjugate according to the first aspect.

A third aspect of the invention relates to the insulin conjugate according to the first aspect for use as a medicament.

### BRIEF DESCRIPTION OF THE DRAWINGS

FIG. 1: Discloses the structure of Conjugate 1. The sequences of the A chain (SEQ ID NO: 13) and the B chain (SEQ ID NO: 9) are indicated in three-letter-code.

FIG. 2: Discloses the structure of Conjugate 2. The sequences of the A chain (SEQ ID NO: 11) and the B chain (SEQ ID NO: 9) are indicated in three-letter-code.

FIG. 3: Discloses the structure of Conjugate 3. The sequences of the A chain (SEQ ID NO: 16) and the B chain (SEQ ID NO: 9) are indicated in three-letter-code.

FIG. 4: Discloses the structure of Conjugate 4. The sequences of the A chain (SEQ ID NO: 16) and the B chain (SEQ ID NO: 8) are indicated in three-letter-code.

FIG. 5: Discloses the structure of Conjugate 5. The sequences of the A chain (SEQ ID NO: 17) and the B chain (SEQ ID NO: 3) are indicated in three-letter-code.

FIG. 6: Discloses the structure of Conjugate 6. The sequences of the A chain (SEQ ID NO: 18) and the B chain (SEQ ID NO: 3) are indicated in three-letter-code.

FIG. 7: Discloses the structure of Conjugate 7. The sequences of the A chain (SEQ ID NO: 16) and the B chain (SEQ ID NO: 3) are indicated in three-letter-code.

### DETAILED DESCRIPTION

### Theory Behind and Advantages of the Present Invention

In order to increase the duration of action of a drug, half-life plays a major role. Half-life (t_{1/2}) is proportional to the volume of distribution divided by clearance. In the case of human insulin, clearance is mainly driven by binding to the insulin receptor, internalization and subsequent degradation. But in a clinical steady state situation, not only elimination of the drug plays a role. Speed of absorption is important, too, since it influences during steady state the peak to trough ratio and thereby the absence/occurrence of unwanted side effect such as hypoglycemia.

In pharmacokinetics, steady state refers to the situation where the overall intake of a drug is fairly in dynamic equilibrium with its elimination. In practice, it is generally considered that steady state is reached when a time of 4 to 5 times the half-life for a drug after regular dosing is started. Especially for drugs such as insulin with a small therapeutic index, the peak to trough ratio is of importance. This index is defined as a measure of the relative desirability of a drug for the attaining of a particular medical end that is usually expressed as the ratio of the largest dose producing no toxic symptoms to the smallest dose routinely producing cures. The trough level is the lowest concentration of a drug in the patient's body, the peak level is the highest concentration of a drug in the patient's body during steady state. Therefore, the peak to trough ratio should be as close to one as possible in order to have an optimal therapeutic effect with minimal side effects.

Accordingly, there is a need for insulin conjugates which have not only a long half-life in vivo but also have a slow absorption in order to achieve a minimal risk of hypoglycemic events in combination with an effective glucose lowering.

Surprisingly, it can be shown in the context of the studies underlying the present invention that preferably a substitution at position A14 with aspartic acid or glutamic acid and preferably also at position B16 with glutamic acid and preferably also at position B25 with histidine of human insulin preferably also in combination with at least 4 additional substitutions with basic amino acids such as arginine resulted in a slow onset of action with a very long duration of action. This allows a once a week administration of the insulin conjugate with a minimal risk of hypoglycemias.

The oral delivery of insulins is hampered due to their instability in the gastrointestinal tract and low mucosal penetration. Mucosal penetration is addressed in the invention by introduction of arginines. In this regard, see Uhl et al. Coating of PLA-nanoparticles with cyclic, arginine-rich cell penetrating peptides enables oral delivery of liraglutide. Nanomedicine: Nanotechnology, Biology, and Medicine 24 (2020) 102132. Surprisingly, the preferred insulin conjugates of the present invention having arginine substitutions at positions A5, A15, A18, B27 show advantageous stability in the presence of trypsin.

### Detailed Description of the First Aspect

The first aspect of the invention relates to an insulin conjugate comprising a human serum albumin binder of Formula (I) and a human insulin analog, wherein the human serum albumin binder of Formula (I) is covalently bound to the human insulin analog in that the terminal carboxy group “a” of the human serum albumin binder of Formula (I) is covalently bound via an amide bond to the epsilon amino group of lysine B29 of the human insulin analog.

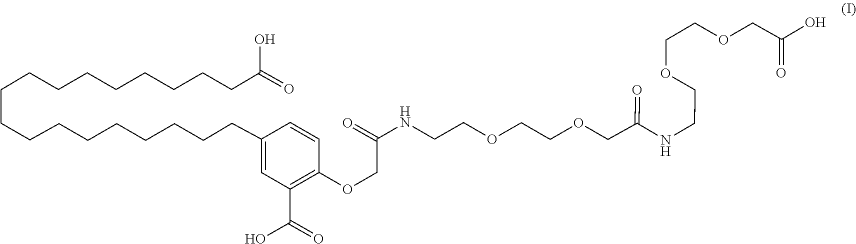 [SMILES: O=C(O)CCCCCCCCCCCCCCCCCCc1ccc(OCC(=O)NCCOCCOCC(=O)NCCOCCOCC(=O)O)c(C(=O)O)c1]

The expression “insulin analog” as used herein refers to a peptide which has a molecular structure which formally can be derived from the structure of a naturally occurring insulin (herein also referred to as “parent insulin”, e.g. human insulin), preferably by deleting and/or substituting at least seven amino acid residues occurring in the naturally occurring insulin. The insulin analog has a human serum albumin binder of Formula (I) attached to the epsilon amino group of lysine B29. The added and/or exchanged amino acid residues can either be codeable amino acid residues or other naturally occurring residues or purely synthetic amino acid residues. The insulin conjugate as referred to herein is capable of lowering blood glucose levels in vivo, such as in a human subject.

In at least one embodiment, the human insulin analog provided herein comprises two peptide chains, an A-chain and a B-chain. Typically, the two chains are connected by disulfide bridges between cysteine residues. For example, in at least one embodiment, the human insulin analog comprises three disulfide bridges: one disulfide bridge between the cysteines at position A6 and A11, one disulfide bridge between the cysteine at position A7 of the A-chain and the cysteine at position B7 of the B-chain, and one between the cysteine at position A20 of the A-chain and the cysteine at position B19 of the B-chain. In at least one embodiment, the human insulin analog comprises cysteine residues at positions A6, A7, A11, A20, B7 and B19. The human serum albumin binder of Formula (I) is attached to the epsilon amino group of lysine B29 of the human insulin analog.

Mutations of human insulin, i.e. mutations of a parent insulin, are indicated herein by referring to the chain, i.e. either the A-chain or the B-chain of the conjugate, the position of the mutated amino acid residue in the A- or B-chain (such as A14, B16 and B25), and the three letter code for the amino acid substituting the native amino acid in the parent insulin. For example, Arg(A5)Glu(A14)Arg(A15)Arg(A18)Gly(A21)Glu(B16)His(B25)Arg(B27)Arg(B31)-human insulin, is an analog of human insulin in which the amino acid residue at position 5 of the A-chain (A5) of human insulin is substituted with arginine, the amino acid residue at position 14 of the A-chain (A14) of human insulin is substituted with glutamic acid, the amino acid residue at position 15 of the A-chain (A15) of human insulin is substituted with arginine, the amino acid residue at position 18 of the A-chain (A18) of human insulin is substituted with arginine, the amino acid residue at position 21 of the A-chain (A21) is substituted with glycine, the amino acid residue at position 16 of the B-chain (B16) is substituted with glutamic acid, the amino acid residue at position 25 of the B-chain (B25) of human insulin is substituted with histidine, the amino acid residue at position 27 of the B-chain (B27) of human insulin is substituted with arginine, and to the C-terminus of the B chain, position 31 (B31) is added with an arginine. The term “desB30” refers to an conjugate lacking the B30 amino acid parent insulin (i.e. the amino acid residue at position B30 is absent).

In some embodiments, the human insulin analog comprises at least one mutation relative to the parent insulin, wherein the insulin conjugate comprises a mutation at position B16 which is substituted with a hydrophobic amino acid, and/or a mutation at position B25 which is substituted with a hydrophobic amino acid. The human insulin analog may optionally comprise further mutations. For example, the amino acid residue at position 14 of the A-chain (A14) of the parent insulin (such as human insulin) may be substituted with glutamic acid, and the amino acid at position 30 of the B chain may be deleted, i.e. is absent (desB30 mutation).

In another aspect herein, the insulin conjugates are long-acting and for administration once a week, for example oral administration.

In at least one embodiment, the insulin conjugates is in the form of a pharmaceutically acceptable salt. Pharmaceutically acceptable salts of the insulin conjugates may include acid addition and base salts. Suitable acid addition salts are formed from acids which form non-toxic salts. In at least one embodiment, the pharmaceutically acceptable salt is selected from the group consisting of acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts. Suitable base salts are formed from bases which form non-toxic salts. Examples include the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-amino-2-(hydroxymethyl)propane-1,3-diol (tris or tromethamine) and zinc salts. Hemisalts of acids and bases may also be formed, for example, hemisulfate and hemicalcium salts. For a review on suitable salts, see the Handbook of Pharmaceutical Salts: Properties, Selection, and Use by Stahl and Wermuth (Wiley-VCH, 2002).

Optionally, the insulin conjugates, and pharmaceutically acceptable salts thereof, may exist in unsolvated and solvated forms. The term ‘solvate’ is used herein to describe a molecular complex comprising the compound of Formula (I), or a pharmaceutically acceptable salt thereof, and one or more pharmaceutically acceptable solvent molecules, for example, ethanol. The term ‘hydrate’ is employed when said solvent is water.

Examples of isotopes suitable for inclusion in the conjugates herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulfur, such as ³⁵S.

Certain isotopically-labelled insulin conjugates, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled conjugates can generally be prepared by conventional techniques known to those skilled in the art.

Pharmaceutically acceptable solvates in accordance with the invention include those wherein the solvent of crystallization may be isotopically substituted, e.g. D₂O, d₆-acetone, d₆-DMSO.

The human insulin analogs provided herein preferably comprise at least seven mutations (substitution, deletion, or addition of an amino acid) relative to parent insulin. The term “at least seven”, as used herein means seven, or more than seven, such as “at least eight”, “at least nine”, etc. In another embodiment, the human insulin analogs provided herein comprise at least two mutations in the B-chain and at least four mutations in the A-chain. For example, the human insulin analogs may comprise a substitution at position B3, B16, B25 and B27, a deletion at position B30 and a substitution at position A5, A9, A14, A18 and A21. Alternatively, the human insulin analog may comprise a substitution at positions B16, B25 and B27, and a substitution at positions A5, A14, A15, A21. Further, the human insulin analog may comprise a substitution at positions B16, B25, B27, an additional amino acid at the C-terminus of the B-chain (B31), and a substitution at positions A5, A14, A15, A18 and A21.

The human insulin analogs provided herein may comprise mutations in addition to the mutations above. In some embodiments, the number of mutations does not exceed a certain number. In some embodiments, the insulin conjugates comprise less than twelve mutations (i.e. deletions, substitution, additions) relative to the parent insulin. In another embodiment, the human insulin analog comprises less than eleven mutations relative to the parent insulin. In another embodiment, the human insulin analog comprises less than ten mutations relative to the parent insulin.

The expression “parent insulin” as used herein refers to naturally occurring insulin, i.e. to an unmutated insulin also known as wildtype human insulin.

The sequence of human insulin is well known in the art and shown in the Example section. Human insulin comprises an A chain having an amino acid sequence GIVEQCCTSICSLYQLENYCN (SEQ ID NO: 1) and a B chain having an amino acid sequence as shown in FVNQHLCGSHLVEALYLVCGERGFFYTPKT (SEQ ID NO: 2).

Human insulin comprises three disulfide bridges: one disulfide bridge between the cysteines at position A6 and A11, one disulfide bridge between the cysteine at position A7 of the A-chain and the cysteine at position B7 of the B-chain, and one between the cysteine at position A20 of the A-chain and the cysteine at position B19 of the B-chain.

The insulin receptor can be any mammalian insulin receptor, such as a bovine, porcine or human insulin receptor. In some embodiments, the insulin receptor is a human insulin receptor, e.g. human insulin receptor isoform A or human insulin receptor isoform B (which was used in the Examples section).

Advantageously, the insulin conjugates provided herein have a later tₘₐₓ in comparison to the insulin conjugate provided in WO15052088 A1 (Madsen, Tagmose, et al., NOVO NORDISK A/S) and at least the same half-life, and a longer half-life than insulin conjugates provided in WO14009316 (Pridal, et al., NOVO NORDISK A/S). tₘₐₓ is the time at which the Cₘₐₓ is observed. Cₘₐₓ is the maximum (or peak) serum concentration that the insulin conjugate achieves. Additionally, they have a very long half-life providing a favourable peak to trough ratio.

Methods for determining the binding affinity of an insulin conjugate to an insulin receptor are well known in the art. For example, the insulin receptor binding affinity can be determined by a scintillation proximity assay which is based on the assessment of competitive binding between [125I]-labelled parent insulin, such as [125I]-labelled human insulin, and the (unlabeled) insulin conjugate to the insulin receptor. The insulin receptor can be present in a membrane of a cell, e.g. of CHO cell, which overexpresses a recombinant insulin receptor. In an embodiment, the insulin receptor binding affinity is determined as described in the Examples section.

Binding of naturally occurring insulin or an insulin conjugate to the insulin receptor activates the insulin signaling pathway. The insulin receptor has tyrosine kinase activity. Binding of insulin to its receptor induces a conformational change that stimulates the autophosphorylation of the receptor on tyrosine residues. The autophosphorylation of the insulin receptor stimulates the receptor's tyrosine kinase activity toward intracellular substrates involved in the transduction of the signal. The autophosphorylation of the insulin receptor by an insulin conjugate is therefore considered as a measure for signal transduction caused by said conjugate.

The insulin receptor autophosphorylation relative to parent insulin can be determined as described in the Examples section.

Typically, the human insulin analog provided herein shows sufficient insulin receptor binding and autophosphorylation activity to elucidate a pharmacological effect. In an embodiment, the human insulin analog shows between 0.5% to 10%, such as 1% to 10% of the insulin receptor binding of the parent insulin and/or 0.2% to 2% of the autophosphorylation activity of the parent insulin (typically human insulin).

In at least one embodiment, the human insulin analog comprises at least seven mutations as compared to the parent insulin. In at least one embodiment, the human insulin analog comprises at least seven mutations, but less than twelve mutations, such as less than eleven mutations as compared to the parent insulin.

In some embodiments insulin conjugates provided herein comprise a human insulin analog having mutations at position A14 which substituted with aspartic acid or glutamic acid, at position B16 which is substituted with glutamic acid or histidine, at position B25 which is substituted with histidine, and at least four additional substitutions. This insulin conjugate can have additional amino acids attached to the human insulin analog. For example, the human insulin analog can have additional arginine residues, such as one or two arginine residues, attached to the human insulin analog. For example, it can have two arginine residues attached to the C-terminus of the B-chain. In an embodiment, the additional arginine residues are added to the C-terminus of the B-chain.

In another embodiment, the insulin conjugates provided herein comprise a human insulin analog having mutations at position A14 which substituted with aspartic acid or glutamic acid, at position B16 which is substituted with aspartic acid or glutamic acid, at position B25 which is substituted with histidine, and at least four additional substitutions. All additional substitutions are arginines, histidines or glycines.

In another embodiment, the insulin conjugates provided herein comprise a human insulin analog having mutations at position A14 which is substituted with aspartic acid or glutamic acid, at position B16 which is substituted with aspartic acid or glutamic acid, at position B25 which is substituted with histidine, at position A21 which is substituted with glycine or arginine, and at least three additional substitutions. In at least one embodiment, all additional substitutions are arginines. In at least one embodiment, the insulin conjugate has additional amino acids attached to the human insulin analog. For example, the human insulin analog can have additional arginine residues, such as one or two arginine residues, attached to the human insulin analog. For example it can have to arginine residues attached to the C-terminus of the B-chain. In an embodiment, the additional arginine residues are added to the C-terminus of the B-chain.

In another embodiment insulin conjugates provided herein comprises a human insulin analog having mutations at position A14 which substituted with aspartic acid or glutamic acid, at position B16 which is substituted with glutamic acid, at position B25 which is substituted with histidine, at position A21 which is substituted with glycine or arginine, and at least three additional substitutions. In at least one embodiment, all additional substitutions are arginines. In at least one embodiment, the insulin conjugate has additional amino acids attached to the insulin chain or chains of the human insulin analog. For example, the human insulin analog can have additional arginine residues, such as one or two arginine residues, attached to the human insulin analog. For example, it can have to arginine residues attached to the C-terminus of the B-chain. In an embodiment, the additional arginine residues are covalently bound to the C-terminus of the B-chain.

At least one embodiment relates to an insulin conjugate comprising a human insulin analog having at least seven mutations relative to parent insulin, optionally wherein the mutations are selected from the group consisting of a substitution, deletion, and an addition of an amino acid residue.

At least one embodiment relates to an insulin conjugate, wherein the insulin conjugate comprises a human insulin analog having mutations:
at position A14 which is substituted with aspartic acid or glutamic acid, at position B16 which is substituted with histidine or glutamic acid,
at position B25 which is substituted with histidine,
and at least four additional substitutions,
and optionally the insulin conjugate has additional amino acid residues attached to the human insulin analog.

At least one embodiment relates to an insulin conjugate, wherein the insulin conjugate comprises a human insulin analog having mutations:
at position A14 which is substituted with aspartic acid or glutamic acid,
at position B16 which is substituted with glutamic acid,
at position B25 which is substituted with histidine,
and at least four additional substitutions,
optionally wherein all additional substitutions are to arginines, to glycines, or a combination thereof,
optionally wherein the insulin conjugate has additional amino acid residues attached to the human insulin analog.

At least one embodiment relates to an insulin conjugate, wherein the insulin conjugate comprises a human insulin analog having mutations
at position A14 which substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the insulin conjugate has additional amino acid residues attached to the human insulin analog.

At least one embodiment relates to an insulin conjugate, wherein the insulin conjugate comprises a human insulin analog having mutations:
at position A14 which is substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the human insulin analog has additional arginine residues attached to the human insulin analog.

In an embodiment, the additional amino acids are additional arginine residues, such as one or two additional arginine residues, attached to the human insulin analog. In an embodiment, the additional arginine residues are attached, i.e. covalently bound, to the C-terminus of the B-chain.

In an embodiment, the B chain of the human insulin analog comprises or consists of the amino acid sequence FVRQHLCGSHLVEALELVCGERGFHYTPK (SEQ ID NO: 3).

In another embodiment, the B chain of the human insulin analog comprises or consists of the amino acid sequence FVRQHLCGSHLVEALELVCGERGFHYRPK (SEQ ID NO: 4).

In another embodiment, the B chain of the human insulin analog comprises or consists of the amino acid sequence FVNQHLCGSHLVEALELVCGERGFHYRPK (SEQ ID NO: 5).

In another embodiment, the B chain of the human insulin analog comprises or consists of the amino acid sequence FVNQHLCGSHLVEALELVCGERGFHYTPK (SEQ ID NO: 6).

In another embodiment, the B chain of the human insulin analog comprises or consists of the amino acid sequence FVNQHLCGSHLVEALHLVCGERGFHYTPK (SEQ ID NO: 7).

In another embodiment, the B chain of the human insulin analog comprises or consists of the amino acid sequence FVNQHLCGSHLVEALELVCGERGFHYTPKTR (SEQ ID NO: 8).

In another embodiment, the B chain of the human insulin analog comprises or consists of the amino acid sequence FVNQHLCGSHLVEALELVCGERGFHYRPKTR (SEQ ID NO: 9).

In another embodiment, the B chain of the human insulin analog comprises or consists of the amino acid sequence FVNQHLCGSHLVEALHLVCGERGFHYRPKTR (SEQ ID NO: 10).

In an embodiment, the A chain of the human insulin analog comprises or consists of the amino acid sequence GIVERCCTSICSLERLERYCG (SEQ ID NO: 11).

In another embodiment, the A chain of the human insulin analog comprises or consists of the amino acid sequence GIVEQCCTSICSLERLERYCR (SEQ ID NO: 12).

In another embodiment, the A chain of the human insulin analog comprises or consists of the amino acid sequence GIVERCCTSICSLEQLERYCG (SEQ ID NO: 13).

In another embodiment, the A chain of the human insulin analog comprises or consists of the amino acid sequence GIVERCCTSICSLEQLERYCR (SEQ ID NO: 14).

In another embodiment, the A chain of the human insulin analog comprises or consists of the amino acid sequence GIVERCCTSICSLERLENYCR (SEQ ID NO: 15).

In another embodiment, the A chain of the human insulin analog comprises or consists of the amino acid sequence GIVERCCTSICSLERLERYCR (SEQ ID NO: 16).

In another embodiment, the A chain of the human insulin analog comprises or consists of the amino acid sequence GIVERCCTRICSLERLERYCR (SEQ ID NO: 17).

In another embodiment, the A chain of the human insulin analog comprises or consists of the amino acid sequence GIVERCCTRICSLERLERYCG (SEQ ID NO: 18).

In another embodiment, the A chain of the human insulin analog comprises or consists of the amino acid sequence GIVEQCCTRICSLERLERYCR (SEQ ID NO: 19).

In another embodiment, the A chain of the human insulin analog comprises or consists of the amino acid sequence GIVERCCTRICSLEQLERYCR (SEQ ID NO: 20).

In another embodiment, the A chain of the human insulin analog comprises or consists of the amino acid sequence GIVERCCTRICSLERLENYCR (SEQ ID NO: 21).

In another embodiment, the A chain of the human insulin analog comprises or consists of the amino acid sequence GIVERCCTSICSLDRLERYCG (SEQ ID NO: 22).

In another embodiment, the A chain of the human insulin analog comprises or consists of the amino acid sequence GIVERCCTSICSLDRLERYCG (SEQ ID NO: 23).

In another embodiment, the A chain of the human insulin analog comprises or consists of the amino acid sequence GIVERCCTSICSLDQLERYCG (SEQ ID NO: 24).

In another embodiment, the A chain of the human insulin analog comprises or consists of the amino acid sequence GIVEQCCTSICSLDRLERYCR (SEQ ID NO: 25).

In another embodiment, the A chain of the human insulin analog comprises or consists of the amino acid sequence GIVERCCTSICSLDQLERYCR (SEQ ID NO: 26).

In another embodiment, the A chain of the human insulin analog comprises or consists of the amino acid sequence GIVERCCTSICSLDRLENYCR (SEQ ID NO: 27).

In another embodiment, the A chain of the human insulin analog comprises or consists of the amino acid sequence GIVERCCTSICSLDRLERYCR (SEQ ID NO: 28).

In another embodiment, the A chain of the human insulin analog comprises or consists of the amino acid sequence GIVERCCTRICSLDRLERYCR (SEQ ID NO: 29).

In another embodiment, the A chain of the human insulin analog comprises or consists of the amino acid sequence GIVERCCTRICSLDRLERYCG (SEQ ID NO: 30).

In another embodiment, the A chain of the human insulin analog comprises or consists of the amino acid sequence GIVEQCCTRICSLDRLERYCR (SEQ ID NO: 31).

In another embodiment, the A chain of the human insulin analog comprises or consists of the amino acid sequence GIVERCCTRICSLDQLERYCR (SEQ ID NO: 32).

In another embodiment, the A chain of the human insulin analog comprises or consists of the amino acid sequence GIVERCCTRICSLDRLENYCR (SEQ ID NO: 33).

Derivatives of the aforementioned amino acids are known in the art.

In at least one embodiment, the insulin conjugate comprises a human insulin analog comprises an A-chain and a B-chain as set forth above.

In at least one embodiment, the human insulin analog is selected from the group consisting of:
Arg(A5)Glu(A14)Arg(A15)Arg(A18)Arg(A21)Glu(B16)His(B25)Arg(B27)Arg(B31)-insulin (human insulin);
Arg(A5)Glu(A14)Arg(A15)Arg(A18)Gly(A21)Glu(B16)His(B25)Arg(B27)Arg(B31)-insulin (human insulin);
Glu(A14)Arg(A15)Arg(A18)Arg(A21)Glu(B16)His(B25)Arg(B27)Arg(B31)-insulin (human insulin);
Arg(A5)Glu(A14)Arg(A18)Gly(A21)Glu(B16)His(B25)Arg(B27)Arg(B31)-insulin (human insulin);
Arg(A5)Glu(A14)Arg(A18)Arg(A21)Glu(B16)His(B25)Arg(B27)Arg(B31)-insulin (human insulin);
Arg(A5)Glu(A14)Arg(A15)Arg(A21)Glu(B16)His(B25)Arg(B27)Arg(B31)-insulin (human insulin);
Arg(A5)Glu(A14)Arg(A15)Arg(A18)Arg(A21)Glu(B16)His(B25)Arg(B31)-insulin (human insulin).

In another embodiment, the human insulin analog is selected from the group consisting of:
Arg(A5)Arg(A9)Glu(A14)Arg(A15)Arg(A18)Arg(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A15)Arg(A18)Arg(A21)Arg(B3)Glu(B16)His(B25)Des(B30)-insulin (human insulin);
Arg(A9)Glu(A14)Arg(A15)Arg(A18)Gly(A21)Arg(B3)Glu(B16)His(B25)Arg(B27)Des(B30)-insulin (human insulin);
Arg(A5)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A18)Gly(A21)Arg(B3)Glu(B16)His(B25)Arg(B27)Des(B30)-insulin (human insulin);
Arg(A5)Arg(A9)Glu(A14)Arg(A15)Gly(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Des(B30)-insulin (human insulin);
Arg(A9)Glu(A14)Arg(A15)Arg(A18)Arg(A21)Arg(B3)Glu(B16)His(B25)Des(B30)-insulin (human insulin);
Arg(A5)Glu(A14)Arg(A15)Arg(A18)Arg(A21)Arg(B3)Glu(B16)His(B25)Des(B30)-insulin (human insulin);
Arg(A5)Arg(A9)Glu(A14)Arg(A18)Arg(A21)Arg(B3)Glu(B16)His(B25)Des(B30)-insulin (human insulin);
Arg(A5)Arg(A9)Glu(A14)Arg(A15)Arg(A18)Arg(A21)Glu(B16)His(B25)Des(B30)-insulin (human insulin);
Arg(A5)Arg(A9)Asp(A14)Arg(A15)Arg(A18)Arg(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A15)Arg(A18)Arg(A21)Arg(B3)Glu(B16)His(B25)Des(B30)-insulin (human insulin);
Arg(A9)Asp(A14)Arg(A15)Arg(A18)Gly(A21)Arg(B3)Glu(B16)His(B25)Arg(B27)Des(B30)-insulin (human insulin);
Arg(A5)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A18)Gly(A21)Arg(B3)Glu(B16)His(B25)Arg(B27)Des(B30)-insulin (human insulin);
Arg(A5)Arg(A9)Asp(A14)Arg(A15)Gly(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Des(B30)-insulin (human insulin);
Arg(A9)Asp(A14)Arg(A15)Arg(A18)Arg(A21)Arg(B3)Glu(B16)His(B25)Des(B30)-insulin (human insulin);
Arg(A5)Asp(A14)Arg(A15)Arg(A18)Arg(A21)Arg(B3)Glu(B16)His(B25)Des(B30)-insulin (human insulin);
Arg(A5)Arg(A9)Asp(A14)Arg(A18)Arg(A21)Arg(B3)Glu(B16)His(B25)Des(B30)-insulin (human insulin);
Arg(A5)Arg(A9)Asp(A14)Arg(A15)Arg(A18)Arg(A21)Glu(B16)His(B25)Des(B30)-insulin (human insulin).

In at least one embodiment, the human insulin analog is selected from the group consisting of:
Arg(A5)Arg(A9)Glu(A14)Arg(A15)Arg(A18)Arg(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A15)Arg(A18)Arg(A21)Arg(B3)Glu(B16)His(B25)Des(B30)-insulin (human insulin);
Arg(A9)Glu(A14)Arg(A15)Arg(A18)Gly(A21)Arg(B3)Glu(B16)His(B25)Arg(B27)Des(B30)-insulin (human insulin);
Arg(A5)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A18)Gly(A21)Arg(B3)Glu(B16)His(B25)Arg(B27)Des(B30)-insulin (human insulin);
Arg(A5)Arg(A9)Glu(A14)Arg(A15)Gly(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Des(B30)-insulin (human insulin);
Arg(A9)Glu(A14)Arg(A15)Arg(A18)Arg(A21)Arg(B3)Glu(B16)His(B25)Des(B30)-insulin (human insulin);
Arg(A5)Glu(A14)Arg(A15)Arg(A18)Arg(A21)Arg(B3)Glu(B16)His(B25)Des(B30)-insulin (human insulin);
Arg(A5)Arg(A9)Glu(A14)Arg(A18)Arg(A21)Arg(B3)Glu(B16)His(B25)Des(B30)-insulin (human insulin);
Arg(A5)Arg(A9)Glu(A14)Arg(A15)Arg(A18)Arg(A21)Glu(B16)His(B25)Des(B30)-insulin (human insulin);
Arg(A5)Arg(A9)Asp(A14)Arg(A15)Arg(A18)Arg(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A15)Arg(A18)Arg(A21)Arg(B3)Glu(B16)His(B25)Des(B30)-insulin (human insulin);
Arg(A9)Asp(A14)Arg(A15)Arg(A18)Gly(A21)Arg(B3)Glu(B16)His(B25)Arg(B27)Des(B30)-insulin (human insulin);
Arg(A5)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A18)Gly(A21)Arg(B3)Glu(B16)His(B25)Arg(B27)Des(B30)-insulin (human insulin);
Arg(A5)Arg(A9)Asp(A14)Arg(A15)Gly(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Des(B30)-insulin (human insulin);
Arg(A9)Asp(A14)Arg(A15)Arg(A18)Arg(A21)Arg(B3)Glu(B16)His(B25)Des(B30)-insulin (human insulin);
Arg(A5)Asp(A14)Arg(A15)Arg(A18)Arg(A21)Arg(B3)Glu(B16)His(B25)Des(B30)-insulin (human insulin);
Arg(A5)Arg(A9)Asp(A14)Arg(A18)Arg(A21)Arg(B3)Glu(B16)His(B25)Des(B30)-insulin (human insulin);
Arg(A5)Arg(A9)Asp(A14)Arg(A15)Arg(A18)Arg(A21)Glu(B16)His(B25)Des(B30)-insulin (human insulin).

In an embodiment, the human insulin analog comprises an A chain having an amino acid sequence as shown in SEQ ID NO: 13 and a B chain having an amino acid sequence as shown in SEQ ID NO: 9.

In another embodiment, the human insulin analog comprises an A chain having an amino acid sequence as shown in SEQ ID NO: 11 and a B chain having an amino acid sequence as shown in SEQ ID NO: 9.

In another embodiment, the human insulin analog comprises an A chain having an amino acid sequence as shown in SEQ ID NO: 16 and a B chain having an amino acid sequence as shown in SEQ ID NO: 9.

In another embodiment, the human insulin analog comprises an A chain having an amino acid sequence as shown in SEQ ID NO: 16 and a B chain having an amino acid sequence as shown in SEQ ID NO: 8.

In another embodiment, the human insulin analog comprises an A chain having an amino acid sequence as shown in SEQ ID NO: 17 and a B chain having an amino acid sequence as shown in SEQ ID NO: 3.

In another embodiment, the human insulin analog comprises an A chain having an amino acid sequence as shown in SEQ ID NO: 18 and a B chain having an amino acid sequence as shown in SEQ ID NO: 3.

In another embodiment, the human insulin analog comprises an A chain having an amino acid sequence as shown in SEQ ID NO: 16 and a B chain having an amino acid sequence as shown in SEQ ID NO: 3.

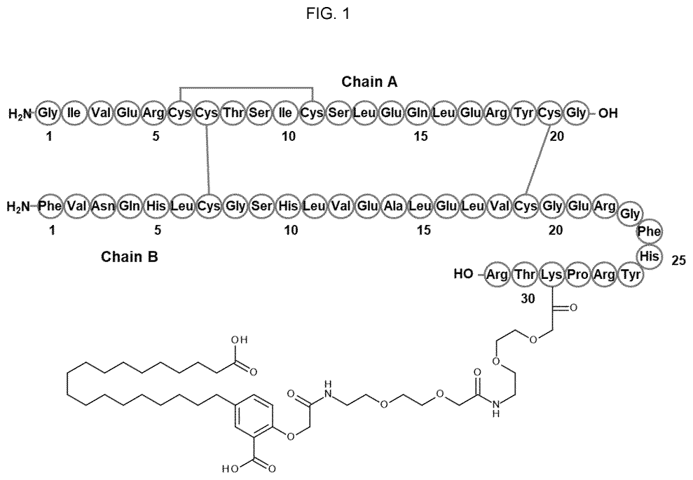

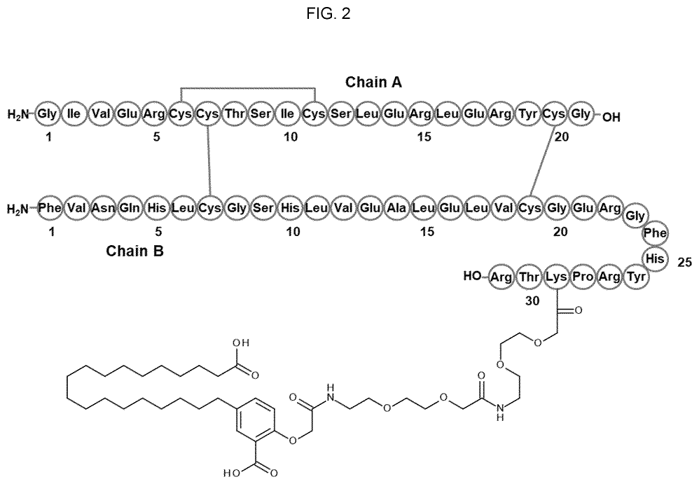

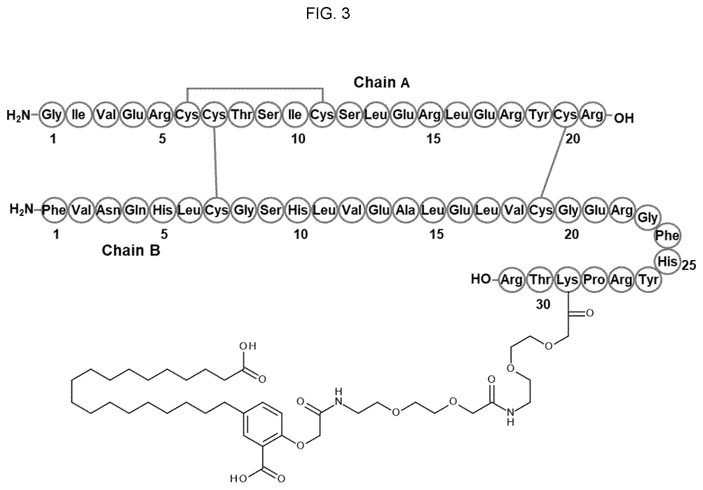

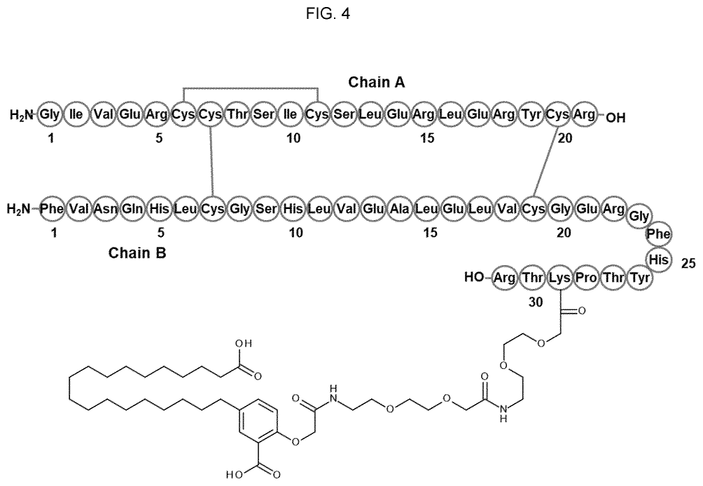

In at least one embodiment, the insulin conjugate is Conjugate 1 according to FIG. 1 or Conjugate 2 according to FIG. 2 or Conjugate 3 according to FIG. 3 or Conjugate 4 according to FIG. 4.

To be clear

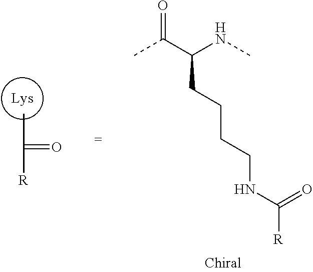

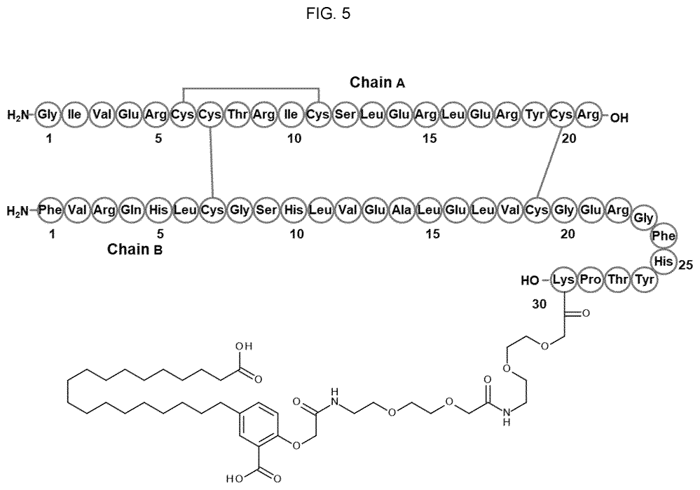

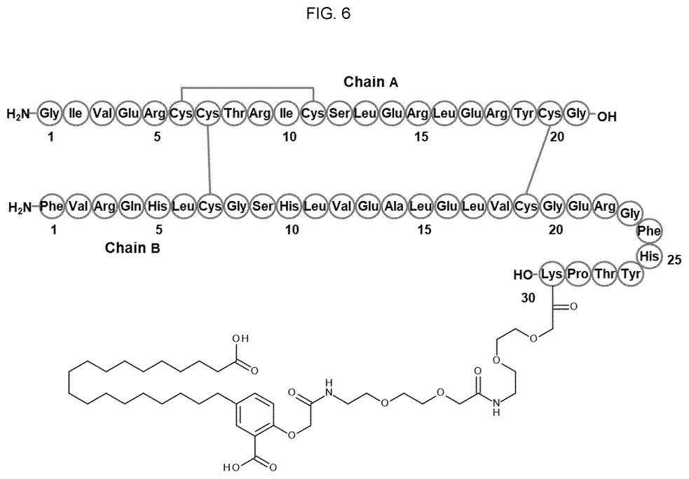

In at least one embodiment, the insulin conjugate is Conjugate 5 according to FIG. 5 or Conjugate 6 according to FIG. 6.

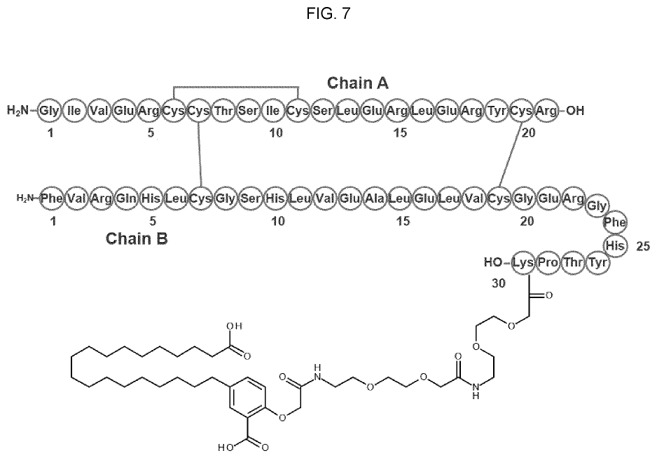

In at least one embodiment, the insulin conjugate is Conjugate 7 according to FIG. 7.

The first aspect further relates to the human insulin analog as defined in connection the with the insulin conjugate of the present invention, for example to the insulin analog as defined in any one of items 3 to 10, such as items 5 to 10, such as items 7 to 10. The items can be found in the section prior to the Examples section. The definitions and explanations for the insulin analog provided for the conjugate, typically, apply mutatis mutandis. In an embodiment, the insulin analog is covalently bound to a moiety which is capable of binding serum albumin, preferably human serum albumin. Serum albumin binding moieties (herein also referred to as “albumin binders” or “binders”), are moieties which when coupled to a peptide such as an insulin analog provided herein, typically, lead to improved pharmacodynamics and/or pharmacokinetic properties of the peptide for example, an extended pharmacokinetic half life in blood and/or blood plasma and/or a prolonged profile of action, i.e. a prolonged reduction of blood glucose level. Typically, the albumin binder is covalently bound to the insulin analog via the epsilon amino group of lysine B29.

### Detailed Description of the Second Aspect

The second aspect relates to a pharmaceutical composition comprising a pharmaceutically effective amount of an insulin conjugate according to the first aspect. The second aspect further relates to a pharmaceutical composition comprising a pharmaceutically effective amount of a human insulin analog according to the first aspect. In at least one embodiment, the pharmaceutical composition comprises a pharmaceutically acceptable excipient.

In at least one embodiment, the pharmaceutical composition comprises zinc ions. In at least one embodiment, the pharmaceutical composition comprises a buffer. In at least one embodiment, the pharmaceutical composition comprises a surfactant. In at least one embodiment, the pharmaceutical composition comprises glycerol. In at least one embodiment, the pharmaceutical composition comprises water.

In at least one embodiment, the pharmaceutical composition has a pH of between pH 3 and pH 8, preferably between pH 3.5 and pH 6.

### Pharmaceutical Composition Example

1 ml pharmaceutical composition consists of 4.2 mmol Conjugate 1, zinc chloride, metacresol (Ph.Eur.), glycerol, hydrochloric acid (pH-value adjustment to pH 4), Polysorbate 20, sodium hydroxide (pH-value adjustment to pH 4), water ad injectabilia.

Other insulin formulations known in the art can be employed herein as pharmaceutical compositions according to the present invention.

In at least one embodiment, the pharmaceutical composition comprises an insulin conjugate in the form of a pharmaceutically acceptable salt. Pharmaceutically acceptable salts of the insulin conjugates may include acid addition and base salts. Suitable acid addition salts are formed from acids which form non-toxic salts. In at least one embodiment, the pharmaceutically acceptable salt is selected from the group consisting of acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts. Suitable base salts are formed from bases which form non-toxic salts. Examples include the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-amino-2-(hydroxymethyl)propane-1,3-diol (tris or tromethamine) and zinc salts. Hemisalts of acids and bases may also be formed, for example, hemisulfate and hemicalcium salts. For a review on suitable salts, see the Handbook of Pharmaceutical Salts: Properties, Selection, and Use by Stahl and Wermuth (Wiley-VCH, 2002).

### Detailed Description of the Third Aspect

The third aspect relates to the insulin conjugate according to the first aspect for use as a medicament. Moreover, the third aspect relates to the human insulin analog according to the first aspect for use as a medicament.

An embodiment relates to the insulin conjugate according to the first aspect for use as a medicament for treatment of a disease selected from the group consisting of gestational diabetes, diabetes mellitus type 1, diabetes mellitus type 2, and hyperglycemia and/or for lowering blood glucose levels.

An embodiment relates to the pharmaceutical composition according to the second aspect for use as a medicament.

An embodiment relates to the pharmaceutical composition according to the second aspect for use as a medicament for treatment of a disease selected from the group consisting of gestational diabetes, diabetes mellitus type 1, diabetes mellitus type 2, and hyperglycemia and/or for lowering blood glucose levels.

### Detailed Description of the Fourth Aspect

A fourth aspect relates to a method of treating a patient comprising administering the insulin conjugate of the first aspect or the pharmaceutical composition of the second aspect to the patient. Further, the fourth aspect relates to a method of treating a patient comprising administering the human insulin analog of the first aspect or the pharmaceutical composition of the second aspect to the patient.

In at least one embodiment, the patient is administered once per week, for example by oral administration.

Provided herein are methods for treating a patient having a disease comprising administering a pharmaceutically effective amount of one or more insulin conjugates provided herein or the pharmaceutical composition thereof to the patient.

In some embodiments, the disease is diabetes mellitus such as diabetes type II mellitus. In at least one embodiment, the disease selected from the group consisting of gestational diabetes, diabetes mellitus type 1, diabetes mellitus type 2, and hyperglycemia and/or for lowering blood glucose levels.

### Detailed Description of the Fifth Aspect

A fifth aspect relates to a proinsulin comprising an insulin A chain as disclosed in the first aspect and/or an insulin B chain as disclosed in the first aspect.

Also provided herein are proinsulins comprising an insulin A chain and/or an insulin B chain of the insulin conjugates provided herein. The B chain may be any B chain as defined herein above for the insulin conjugates provided herein. The insulin B chain may comprise further mutations as described herein above for the B chain.

The A chain comprised by the proinsulin provided herein may be any A chain as defined herein above for the insulin conjugates provided herein.

In addition to the insulin A chain and/or the insulin B chain, the proinsulins provided herein may comprise further elements such as leader sequences or a C-peptide. In some embodiments, the proinsulin may further comprise a C-peptide which is located between the insulin B chain and the insulin A chain. The C-peptide may have a length of 0-15 amino acids. In at least one embodiment, the proinsulin does not comprise a C-peptide. The orientation may be as follows (from N-terminus to C-terminus): B chain, C-peptide, A chain.

### Detailed Description of the Sixth Aspect

A sixth aspect of the present invention relates to a method of preparing the insulin conjugate of the first aspect.

In at least one embodiment, the human serum albumin binder of Formula (I) is covalently attached to the human insulin analog by forming an amide bond between the terminal carboxy group “a” of the human serum albumin binder of Formula (I) and the epsilon amino group of lysine B29 of the human insulin analog.

The insulin conjugate can be prepared by any method deemed appropriate. For example, the insulin conjugate can be prepared by recombinant methods or by solid-phase synthesis.

The definitions and explanations given above apply mutatis mutandis to the sixth aspect.

Provided herein are human insulin B chains, i.e. human insulin B chain peptides, as defined hereinabove in connection with the B chain of the insulin conjugate. Accordingly, provided herein are insulin B chains which comprise at least two mutations relative to the insulin B chain of the parent insulin. The insulin B chain may comprise further mutations as described herein above such as the Des(B30) deletion.

Provided herein are polynucleotides encoding the human insulin analog B chains, and the proinsulins provided herein. Said polynucleotide may be operably linked to a promoter which allows for the expression of said polynucleotide. In some embodiments, the promoter is heterologous with respect to said polynucleotide. In some embodiments, the promoter is a constitutive promoter. In another embodiment, the promoter is an inducible promoter.

Further, provided herein are vectors comprising the polynucleotide encoding the insulin conjugates provided herein. In some embodiments, said vector is an expression vector.

Provided herein are host cells comprising nucleic acids encoding the insulin conjugates, insulin B chains, and proinsulins, the polynucleotides, and/or the vectors provided herein. In some embodiments, the host cell is a bacterial cell such as a cell of belonging to the genus Escherichia, e.g. an E. coli cell. In another embodiment, the host cell is a yeast cell, such as a Komagataella phaffii (sometimes also referred to as “Pichia pastoris”) cell or a Kluyveromyces lactis cell.

Another aspect of the present invention relates to a polynucleotide encoding the human insulin analog according to the first aspect.

Another aspect of the present invention relates to an expression vector comprising the polynucleotide encoding the human insulin analog according to the first aspect.

Another aspect of the present invention relates to a host cell comprising the human insulin analog according to the first aspect, a polynucleotide encoding the human insulin analog according to the first aspect, and/or an expression vector comprising the polynucleotide encoding the human insulin analog according to the first aspect.

### Detailed Description of the Seventh Aspect

A seventh aspect relates to a stable derivative of the human albumin binder of Formula (I)

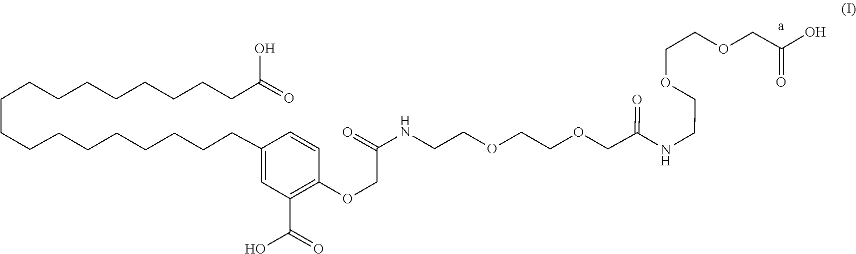 [SMILES: O=C(O)CCCCCCCCCCCCCCCCCCc1ccc(OCC(=O)NCCOCCOCC(=O)NCCOCCOCC(=O)O)c(C(=O)O)c1]

In at least one embodiment, the stable derivative of the human albumin binder of Formula (I) is an ester, preferably wherein “a” is an ester bond.

In at least one embodiment, the stable derivative of the human albumin binder of Formula (I) is an O-succinimide ester.

### Detailed Description of the Eighth Aspect

An eighth aspect relates to the non-therapeutic use of the insulin conjugate or insulin analog according to the first aspect.

### Detailed Description of Certain Embodiments (Clauses and Items)

In the following, an overview on certain embodiments is provided. The definitions above, typically, apply mutatis mutandis to the following clauses and items.

### Clauses

1. An insulin conjugate comprising a human serum albumin binder of Formula (I) and a human insulin analog, wherein the human serum albumin binder of Formula (I) is covalently bound to the human insulin analog in that the terminal carboxy group “a” of the human serum albumin binder of Formula (I) is covalently bound via an amide bond to the epsilon amino group of lysine B29 of the human insulin analog.

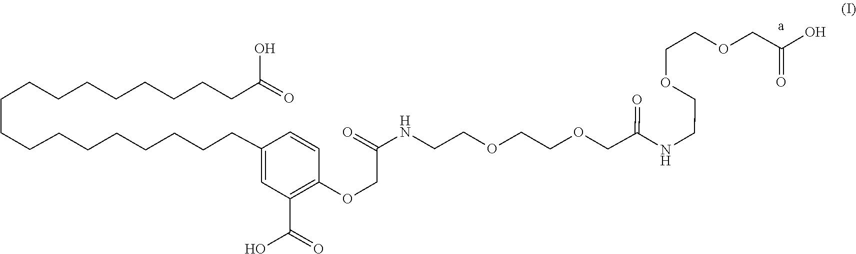 [SMILES: O=C(O)CCCCCCCCCCCCCCCCCCc1ccc(OCC(=O)NCCOCCOCC(=O)NCCOCCOCC(=O)O)c(C(=O)O)c1]

2. The insulin conjugate according to clause 1, comprising a human insulin analog having at least seven mutations relative to parent insulin, optionally wherein the mutations are selected from the group consisting of a substitution, deletion, and an addition of an amino acid residue.

3. The insulin conjugate according to any of the preceding clauses, wherein the insulin conjugate comprises a human insulin analog having mutations:
at position A14 which is substituted with aspartic acid or glutamic acid, at position B16 which is substituted with histidine or glutamic acid,
at position B25 which is substituted with histidine,
and at least four additional substitutions,
and optionally the insulin conjugate has additional amino acid residues attached to the human insulin analog.

4. The insulin conjugate according to any of the preceding clauses, wherein the insulin conjugate comprises a human insulin analog having mutations:
at position A14 which is substituted with aspartic acid or glutamic acid,
at position B16 which is substituted with glutamic acid,
at position B25 which is substituted with histidine,
and at least four additional substitutions,
optionally wherein all additional substitutions are to arginines, to glycines, or a combination thereof,
optionally wherein the insulin conjugate has additional amino acid residues attached to the human insulin analog.

5. The insulin conjugate according to any of the preceding clauses, wherein the insulin conjugate comprises a human insulin analog having mutations
at position A14 which substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the insulin conjugate has additional amino acid residues attached to the human insulin analog.

6. The insulin conjugate according to any of the preceding clauses, wherein the insulin conjugate comprises a human insulin analog having mutations:
at position A14 which is substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the insulin conjugate has additional arginine residues attached to the human insulin analog.

7. The insulin conjugate according to any of the preceding clauses, wherein the insulin conjugate comprises a human insulin analog selected from the group consisting of:
Arg(A5)Glu(A14)Arg(A15)Arg(A18)Arg(A21)Glu(B16)His(B25)Arg(B27)Arg(B31)insulin (human insulin);
Arg(A5)Glu(A14)Arg(A15)Arg(A18)Gly(A21)Glu(B16)His(B25)Arg(B27)Arg(B31)insulin (human insulin);
Arg(A5)Glu(A14)Arg(A18)Gly(A21)Glu(B16)His(B25)Arg(B27)Arg(B31)-insulin (human insulin);
Glu(A14)Arg(A15)Arg(A18)Arg(A21)Glu(B16)His(B25)Arg(B27)Arg(B31)-insulin (human insulin);
Arg(A5)Glu(A14)Arg(A18)Arg(A21)Glu(B16)His(B25)Arg(B27)Arg(B31)-insulin (human insulin);
Arg(A5)Glu(A14)Arg(A15)Arg(A21)Glu(B16)His(B25)Arg(B27)Arg(B31)-insulin (human insulin);
Arg(A5)Glu(A14)Arg(A15)Arg(A18)Arg(A21)Glu(B16)His(B25)Arg(B31)-insulin (human insulin).

8. The insulin conjugate according to any of the preceding clauses, wherein the insulin conjugate comprises a human insulin analog selected from the group consisting of:
Arg(A5)Arg(A9)Glu(A14)Arg(A15)Arg(A18)Arg(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A15)Arg(A18)Arg(A21)Arg(B3)Glu(B16)His(B25)Des(B30)-insulin (human insulin);
Arg(A9)Glu(A14)Arg(A15)Arg(A18)Gly(A21)Arg(B3)Glu(B16)His(B25)Arg(B27)Des(B30)-insulin (human insulin);
Arg(A5)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A18)Gly(A21)Arg(B3)Glu(B16)His(B25)Arg(B27)Des(B30)-insulin (human insulin);
Arg(A5)Arg(A9)Glu(A14)Arg(A15)Gly(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Des(B30)-insulin (human insulin);
Arg(A9)Glu(A14)Arg(A15)Arg(A18)Arg(A21)Arg(B3)Glu(B16)His(B25)Des(B30)-insulin (human insulin);
Arg(A5)Glu(A14)Arg(A15)Arg(A18)Arg(A21)Arg(B3)Glu(B16)His(B25)Des(B30)-insulin (human insulin);
Arg(A5)Arg(A9)Glu(A14)Arg(A18)Arg(A21)Arg(B3)Glu(B16)His(B25)Des(B30)-insulin (human insulin);
Arg(A5)Arg(A9)Glu(A14)Arg(A15)Arg(A18)Arg(A21)Glu(B16)His(B25)Des(B30)-insulin (human insulin);
Arg(A5)Arg(A9)Asp(A14)Arg(A15)Arg(A18)Arg(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A15)Arg(A18)Arg(A21)Arg(B3)Glu(B16)His(B25)Des(B30)-insulin (human insulin);
Arg(A9)Asp(A14)Arg(A15)Arg(A18)Gly(A21)Arg(B3)Glu(B16)His(B25)Arg(B27)Des(B30)-insulin (human insulin);
Arg(A5)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A18)Gly(A21)Arg(B3)Glu(B16)His(B25)Arg(B27)Des(B30)-insulin (human insulin);
Arg(A5)Arg(A9)Asp(A14)Arg(A15)Gly(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Des(B30)-insulin (human insulin);
Arg(A9)Asp(A14)Arg(A15)Arg(A18)Arg(A21)Arg(B3)Glu(B16)His(B25)Des(B30)-insulin (human insulin);
Arg(A5)Asp(A14)Arg(A15)Arg(A18)Arg(A21)Arg(B3)Glu(B16)His(B25)Des(B30)-insulin (human insulin);
Arg(A5)Arg(A9)Asp(A14)Arg(A18)Arg(A21)Arg(B3)Glu(B16)His(B25)Des(B30)-insulin (human insulin);
Arg(A5)Arg(A9)Asp(A14)Arg(A15)Arg(A18)Arg(A21)Glu(B16)His(B25)Des(B30)-insulin (human insulin).

9. The insulin conjugate according to any of the preceding clauses, wherein the insulin conjugate is Conjugate 1, 2, 3, 4, 5, 6 or 7 (see FIGS. 1 to 7).

10. A human insulin analog as defined in any one of clauses 3 to 10, such as clauses 5 to 8.

11. A human insulin analog as defined in clause 7 or 8.

12. A pharmaceutical composition comprising in a pharmaceutically effective amount the insulin conjugate according to any of clauses 1 to 9, or the insulin analog of clause 10 or 11.

13. An insulin conjugate according to any of clauses 1 to 9, or the insulin analog of clause 10 or 11 for use as a medicament.

14. An insulin conjugate according to any of clauses 1 to 9, or the insulin analog of clause 10 or 11 for use as a medicament for the treatment of a disease selected from the group consisting of gestational diabetes, diabetes mellitus type 1, diabetes mellitus type 2, and hyperglycemia and/or for lowering blood glucose levels.

15. An insulin conjugate according to any of clauses 1 to 9, or the insulin analog of clause 10 or 11, for use as a medicament for the treatment of diabetes mellitus type 2.

### Items

1. An insulin conjugate comprising a human serum albumin binder of Formula (I) and a human insulin analog, wherein the human serum albumin binder of Formula (I) is covalently bound to the human insulin analog in that the terminal carboxy group “a” of the human serum albumin binder of Formula (I) is covalently bound via an amide bond to the epsilon amino group of lysine B29 of the human insulin analog.

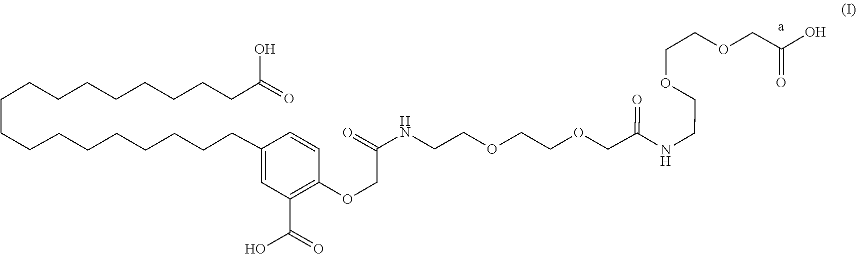 [SMILES: O=C(O)CCCCCCCCCCCCCCCCCCc1ccc(OCC(=O)NCCOCCOCC(=O)NCCOCCOCC(=O)O)c(C(=O)O)c1]

2. The insulin conjugate according to item 1, comprising a human insulin analog having at least seven mutations relative to parent insulin, optionally wherein the mutations are selected from the group consisting of a substitution, deletion, and an addition of an amino acid residue.

3. The insulin conjugate according to any of the preceding items, wherein the insulin conjugate comprises a human insulin analog having mutations:
at position A14 which is substituted with aspartic acid or glutamic acid, at position B16 which is substituted with histidine or glutamic acid,
at position B25 which is substituted with histidine,
and at least four additional substitutions,
and optionally the insulin conjugate has additional amino acid residues attached to the human insulin analog.

4. The insulin conjugate according to any of the preceding items, wherein the insulin conjugate comprises a human insulin analog having mutations:
at position A14 which is substituted with aspartic acid or glutamic acid,
at position B16 which is substituted with glutamic acid,
at position B25 which is substituted with histidine,
and at least four additional substitutions,
optionally wherein all additional substitutions are to arginines, to glycines, or a combination thereof,
optionally wherein the insulin conjugate has additional amino acid residues attached to the human insulin analog.

5. The insulin conjugate according to any of the preceding items, wherein the insulin conjugate comprises a human insulin analog having mutations
at position A14 which substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the insulin conjugate has additional amino acid residues attached to the human insulin analog.

6. The insulin conjugate according to any of the preceding items, wherein the insulin conjugate comprises a human insulin analog having mutations:
at position A14 which is substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the insulin conjugate has additional arginine residues attached to the human insulin analog.

7. The insulin conjugate according to any of the preceding items, wherein the insulin conjugate comprises a human insulin analog selected from the group consisting of:
Arg(A5)Glu(A14)Arg(A15)Arg(A18)Arg(A21)Glu(B16)His(B25)Arg(B27)Arg(B31)insulin (human insulin);
Arg(A5)Glu(A14)Arg(A15)Arg(A18)Gly(A21)Glu(B16)His(B25)Arg(B27)Arg(B31)insulin (human insulin);
Arg(A5)Glu(A14)Arg(A18)Gly(A21)Glu(B16)His(B25)Arg(B27)Arg(B31)-insulin (human insulin);
Glu(A14)Arg(A15)Arg(A18)Arg(A21)Glu(B16)His(B25)Arg(B27)Arg(B31)-insulin (human insulin);
Arg(A5)Glu(A14)Arg(A18)Arg(A21)Glu(B16)His(B25)Arg(B27)Arg(B31)-insulin (human insulin);
Arg(A5)Glu(A14)Arg(A15)Arg(A21)Glu(B16)His(B25)Arg(B27)Arg(B31)-insulin (human insulin);
Arg(A5)Glu(A14)Arg(A15)Arg(A18)Arg(A21)Glu(B16)His(B25)Arg(B31)-insulin (human insulin).

8. The insulin conjugate according to any of the preceding items, wherein the insulin conjugate comprises a human insulin analog selected from the group consisting of:
Arg(A5)Arg(A9)Glu(A14)Arg(A15)Arg(A18)Arg(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A15)Arg(A18)Arg(A21)Arg(B3)Glu(B16)His(B25)Des(B30)-insulin (human insulin);
Arg(A9)Glu(A14)Arg(A15)Arg(A18)Gly(A21)Arg(B3)Glu(B16)His(B25)Arg(B27)Des(B30)-insulin (human insulin);
Arg(A5)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A18)Gly(A21)Arg(B3)Glu(B16)His(B25)Arg(B27)Des(B30)-insulin (human insulin);
Arg(A5)Arg(A9)Glu(A14)Arg(A15)Gly(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Des(B30)-insulin (human insulin);
Arg(A9)Glu(A14)Arg(A15)Arg(A18)Arg(A21)Arg(B3)Glu(B16)His(B25)Des(B30)-insulin (human insulin);
Arg(A5)Glu(A14)Arg(A15)Arg(A18)Arg(A21)Arg(B3)Glu(B16)His(B25)Des(B30)-insulin (human insulin);
Arg(A5)Arg(A9)Glu(A14)Arg(A18)Arg(A21)Arg(B3)Glu(B16)His(B25)Des(B30)-insulin (human insulin);
Arg(A5)Arg(A9)Glu(A14)Arg(A15)Arg(A18)Arg(A21)Glu(B16)His(B25)Des(B30)-insulin (human insulin);
Arg(A5)Arg(A9)Asp(A14)Arg(A15)Arg(A18)Arg(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A15)Arg(A18)Arg(A21)Arg(B3)Glu(B16)His(B25)Des(B30)-insulin (human insulin);
Arg(A9)Asp(A14)Arg(A15)Arg(A18)Gly(A21)Arg(B3)Glu(B16)His(B25)Arg(B27)Des(B30)-insulin (human insulin);
Arg(A5)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A18)Gly(A21)Arg(B3)Glu(B16)His(B25)Arg(B27)Des(B30)-insulin (human insulin);
Arg(A5)Arg(A9)Asp(A14)Arg(A15)Gly(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Des(B30)-insulin (human insulin);
Arg(A9)Asp(A14)Arg(A15)Arg(A18)Arg(A21)Arg(B3)Glu(B16)His(B25)Des(B30)-insulin (human insulin);
Arg(A5)Asp(A14)Arg(A15)Arg(A18)Arg(A21)Arg(B3)Glu(B16)His(B25)Des(B30)-insulin (human insulin);
Arg(A5)Arg(A9)Asp(A14)Arg(A18)Arg(A21)Arg(B3)Glu(B16)His(B25)Des(B30)-insulin (human insulin);
Arg(A5)Arg(A9)Asp(A14)Arg(A15)Arg(A18)Arg(A21)Glu(B16)His(B25)Des(B30)-insulin (human insulin).

9. The insulin conjugate according to any of the preceding items, wherein the insulin conjugate is Conjugate 1 (A chain sequence: SEQ ID NO: 13; B chain sequence: SEQ ID NO: 9):

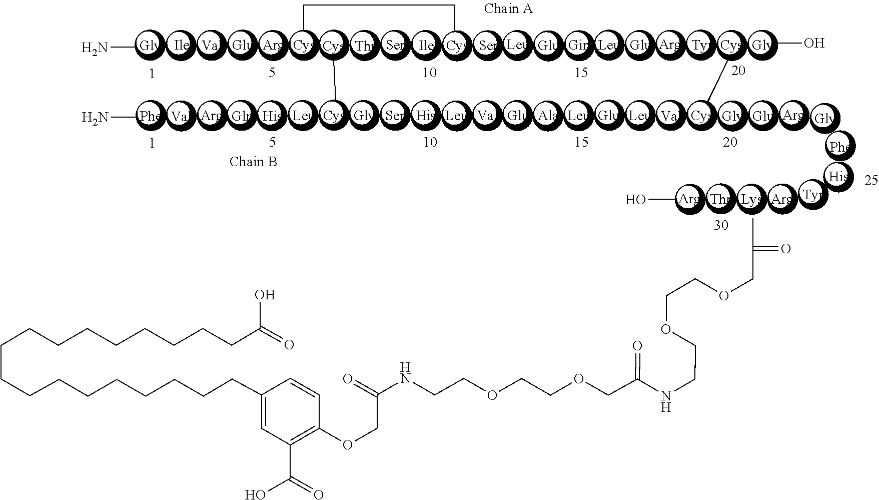

or
Conjugate 2 (A chain sequence: SEQ ID NO: 11; B chain sequence: SEQ ID NO: 9):

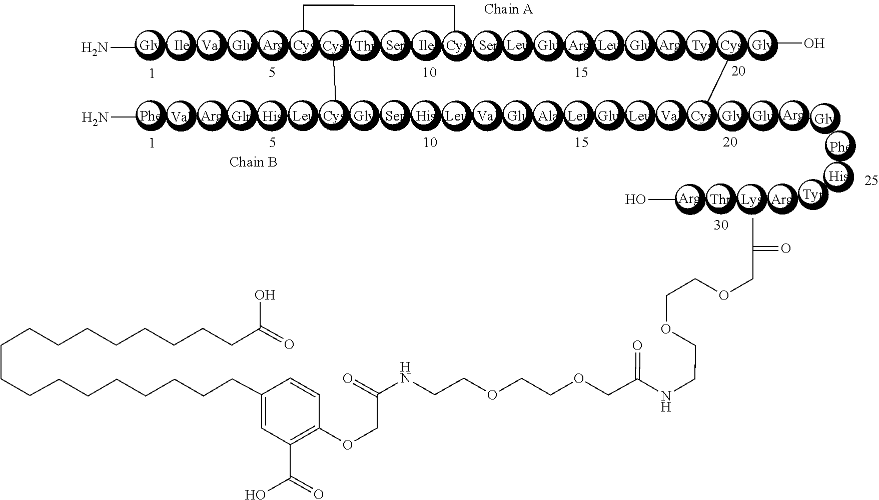

Conjugate 3 (A chain sequence: SEQ ID NO: 16; B chain sequence: SEQ ID NO: 9):

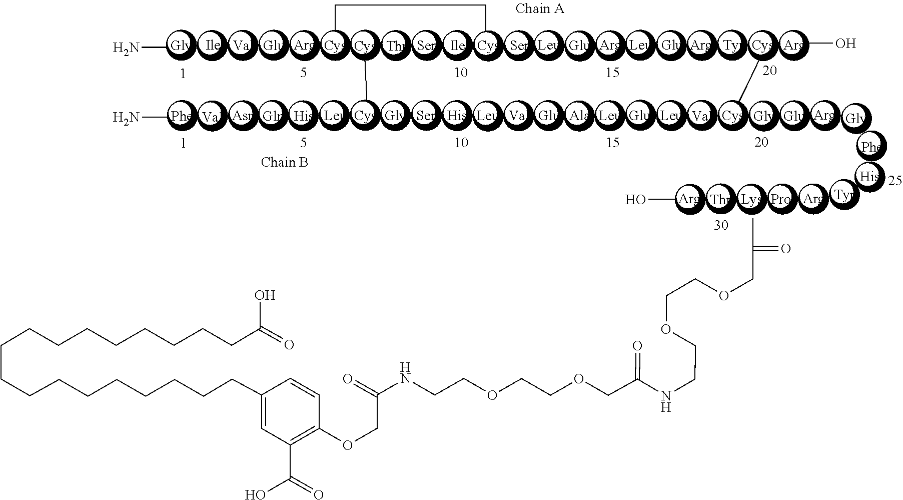

or
Conjugate 4 (A chain sequence: SEQ ID NO: 16; B chain sequence: SEQ ID NO: 8):

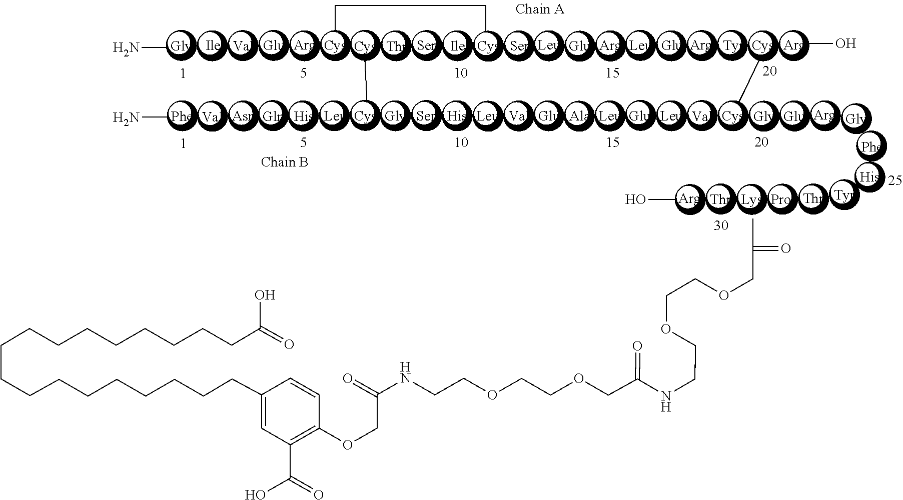

10. The insulin conjugate according to any of the preceding items, wherein the insulin conjugate is Conjugate 5 (A chain sequence: SEQ ID NO: 17; B chain sequence: SEQ ID NO: 3):

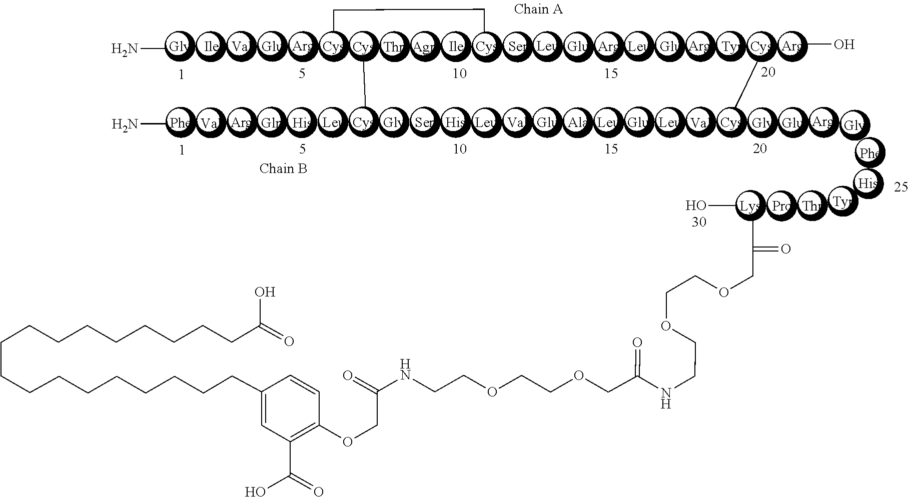

or
Conjugate 6 (A chain sequence: SEQ ID NO: 18; B chain sequence: SEQ ID NO: 3):

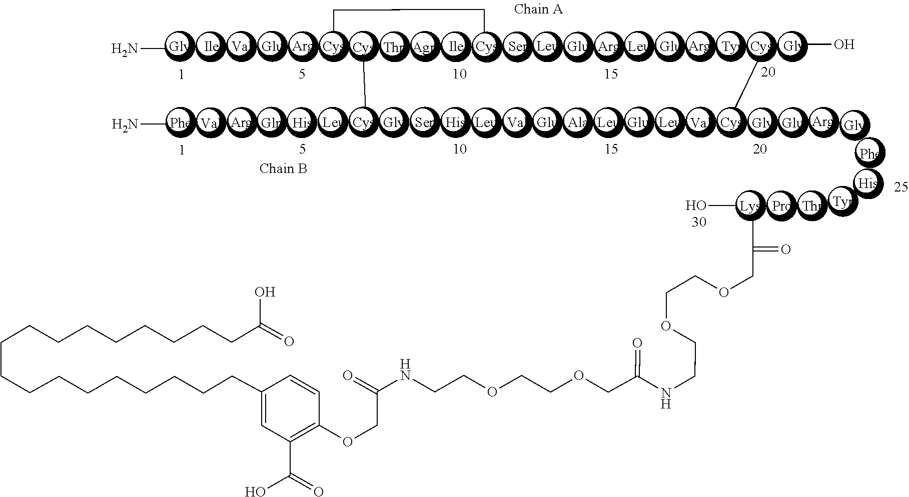

12. An insulin conjugate according to any of items 1 to 10 for use as a medicament.

13. An insulin conjugate according to any of items 1 to 10 for use as a medicament for treatment of a disease selected from the group consisting of gestational diabetes, diabetes mellitus type 1, diabetes mellitus type 2, and hyperglycemia and/or for lowering blood glucose levels.

### SYNTHESIS EXAMPLES

### Synthesis Section 1: Synthesis Human Serum Albumin Binder. In Detail

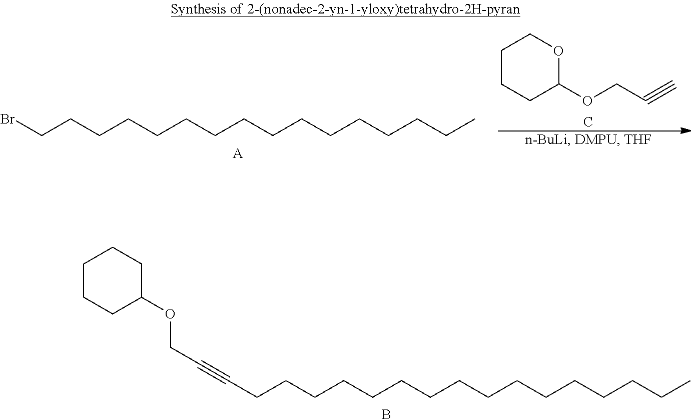

BuLi (2.5 Min hexanes, 30 mL, 75 mmol) was added dropwise to a stirred solution of C (10 g, 72 mmol) in THF (100 mL) at −78° C., and the resultant mixture was stirred at this temperature for 30 min. DMPU (10 g, 78 mmol) was then added, and stirring was continued for a further 10 min. A (10 g, 78 mmol) was then added dropwise, and the resultant mixture was allowed to warm to rt and then heated at 55° C. for 20 h. The mixture was allowed to cool to rt, and saturated aqueous NH₄Cl (200 mL) was added. The layers were separated, and the aqueous layer was extracted with EA (300 mL). The combined organic layers were washed with water and brine, dried over anhydrous Na₂SO₄. The crude product B was used directly without further purification. (9.6 g, 80%).

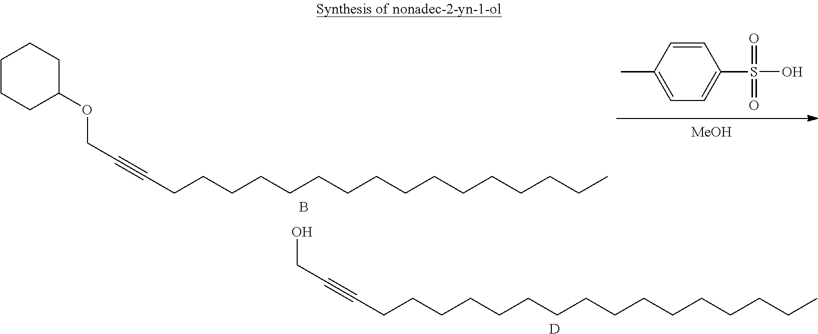

4-methylbenzenesulfonic acid (2 g, 0.4 mmol) was added to B (9.6 g, 26.3 mmol) in MeOH (100 mL) and the reaction mixture was stirred for 12 h. The solvent was removed after hydrolysis with H2O (300 mL), extraction with EA (2×300 mL), and drying over Na₂SO₄, concentrated under the vacuum. The crude was purified by silica gel chromatography (eluting with 5% EA in PE) to afford the desired product D. (6.2 g, 85%) NO LCMS

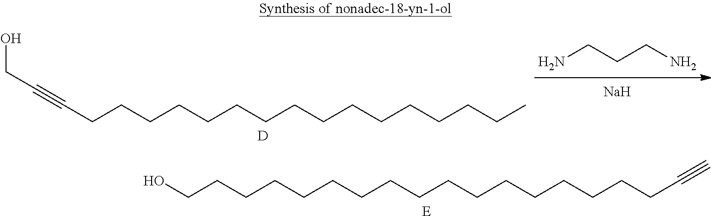

To NaH (60% in mineral oil, 5.7 g, 142.8 mmol, washed free of oil three times with hexane) was added 1,3-Diaminopropane (80 mL). The mixture was stirred in a constant-temperature oil bath at 70° C. After 10 min gas evolution was noted and after 1 h a clear solution of D (4 g, 14.28 mmol) in 1, 3-Diaminopropane (40 mL) was added. The reddish brown mixture was stirred at 55° C. overnight and then cooled, water was added, and the organic product was extracted four times with ether. The combined ether phases were washed successively with water, dilute HCl, and NaCl solutions and then dried over Na₂SO₄, concentrated under the vacuum. The crude was purified by silica gel chromatography (eluting with 10% EA in PE) to afford the desired product E. (2.8 g, 70%)

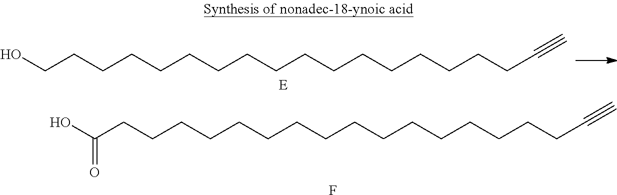

To a solution of E (2.8 g, 10 mmol) and TEMPO (782 mg, 5 mmol) in CH₃CN (20 mL), THF (20 mL) and pH 4-buffer solution (20 mL) was added, NaClO₂ (5 g, 55 mmol) and a 10% solution of NaOCl (372 mg, 5 mmol) simultaneously. The reaction mixture was stirred at RT overnight, diluted with EA (150 mL), washed with water (100 mL) and brine, dried over Na₂SO₄, concentrated under the vacuum. The crude product F was used directly without further purification. (2.5 g, 86%)

¹H NMR (400 MHz, CDCl₃) δ 2.40-2.31 (m, 2H), 2.18 (td, J=7.0, 2.4 Hz, 2H), 1.94 (t, J=2.5 Hz, 1H), 1.69-1.58 (m, 2H), 1.51 (dd, J=14.8, 7.2 Hz, 2H), 1.37-1.18 (m, 24H).

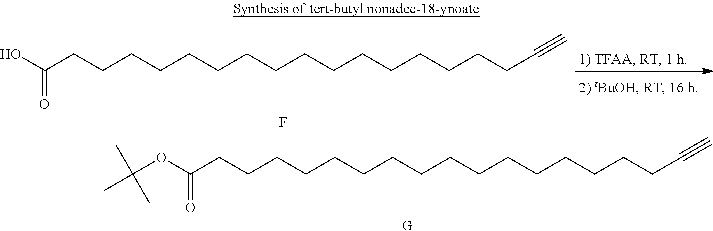

F (1.3 g, 4.42 mmol), TFAA (2.8 g, 13.2 mmol) was added to THF (10 mL), the mixture reacted at room temperature for 1 h. Then t-BuOH (10 mL) was added to the mixture, and stirred for 16 h at room temperature. The pH of reaction mixture was adapted to pH=8 with NaHCO₃ solution, extracted with EA (200 mL*3), dried over Na₂SO₄, concentrated to afford the target compound G. (1.2 g, 80%)

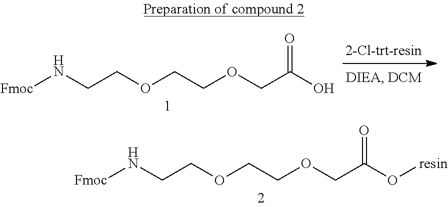

General Procedure: A solution of compound 1 (4.62 g, 12.0 mmol) in anhydrous DCM (60 mL) was added to 2-Cl-trt-resin (5.0 g, 6.0 mmol), then DIEA (1.55 g, 12.0 mmol) was added. The mixture was vortexed at 5-10° C. for 20 hrs. The mixture was filtered, washed with NMP (60 mL*3), DCM (60 mL*3) and NMP (60 mL*3) to give compound 2 (10 g, crude) as yellow solid, which was used in next step directly.

LCMS: WH01668-109-2A m/z 386.2 [M+1]⁺

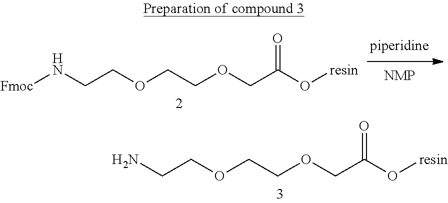

General Procedure: 20% piperidine/NMP (60 mL) was added to compound 2 (10 g, crude, 6.0 mmol in theory). The mixture was vortexed at 5˜10° C. for 30 min, filtered, the residue was treated with the same procedure twice. The residue was washed with NMP (60 mL*3), DCM (60 mL*3) and NMP (60 mL*3) to give compound 3 (7.0 g, crude) as yellow solid, which was used in next step directly. The LCMS showed no compound 2 remained, but the compound 3 wasn't detected. A positive TNBS test gave red-colored resins.

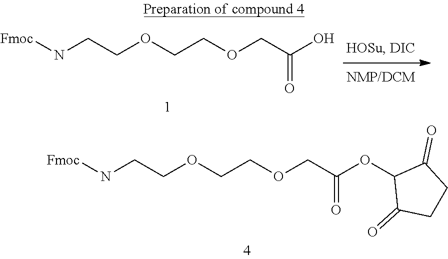

General Procedure: To a solution of compound 1 (4.63 g, 12.0 mmol) in NMP (30 mL) and DCM (30 mL) was added N-hydroxysuccinimide (1.66 g, 14.4 mmol) and Diisopropylcarbodiimide (1.82 g, 14.4 mmol). The mixture was stirred at 5˜10° C. for 4 hrs, TLC showed the reaction was completed. The reaction mixture contained compound 4 was used in next step directly.

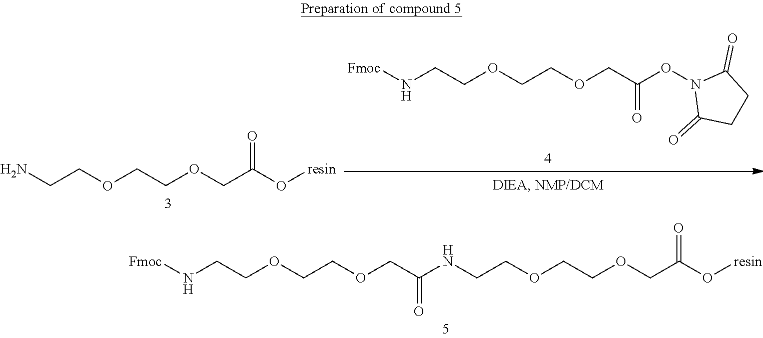

General Procedure: The reaction mixture contained compound 4 (12.0 mmol in theory) was added to compound 3 (7.0 g. crude, 6.0 mmol in theory), then DIEA (1.86 g, 14.4 mmol) was added. The mixture was vortexed at 5˜10° C. for 16 hrs. The mixture was filtered, washed with NMP (60 mL*3), DCM (60 mL*3) and NMP (60 mL*3) to give compound 5 (12.0 g, crude) as yellow solid, which was used in next step directly. A positive TNBS test gave colorless resins.

LCMS: WH01668-112-3A m/z 531.3 [M+1]⁺

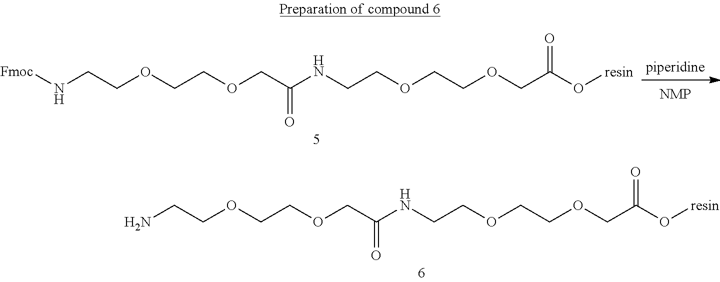

General Procedure: 20% piperidine/NMP (60 mL) was added to compound 5 (10 g, crude, 6.0 mmol in theory). The mixture was vortexed at 5˜10° C. for 30 min, filtered, the residue was treated with the same procedure twice. The residue was washed with NMP (60 mL*3), DCM (60 mL*3) and NMP (60 mL*3) to give compound 6 (7.0 g, crude) as yellow solid, which was used in next step directly. The LCMS showed no compound 5 remained, but the compound 6 wasn't detected. A positive TNBS test gave red-colored resins.

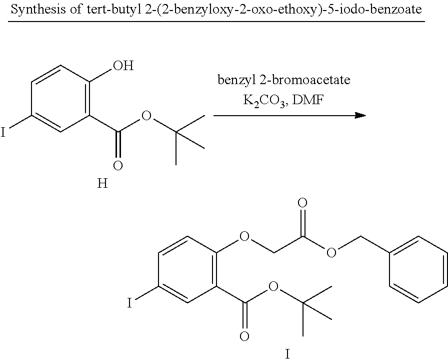

General Procedure: K₂CO₃ (0.86 g, 6 mmol) was added to a solution of H (1 g, 3 mmol) in DMF (10 mL), and the mixture was stirred at room temperature for 5 min. Then benzyl bromoacetate (4.5 mmol, 1.05 g) was added to the mixture, the temperature was raised to 45° C., and the reaction was stirred for 2 h at room temperature. Afterward, the reaction was diluted with 50 mL of EtOAc and washed with brine (3×50 mL). The organic phase was dried over MgSO₄, filtered, and the solvent was removed under reduced pressure. The crude was purified by stirring the mixture with pentane (5 mL). The solid product was collected in a filter to obtain I (0.98 g, 70%) as a yellow-brown solid.

¹H NMR: (400 Mhz, DMSO) δ 7.8 (d, 1H), 7.7 (dd, 1H), 7.38 (s, 5H), 6.9 (d, 1H), 5.21 (s, 2H), 4.92 (s, 2H), 1.5 (s, 9H)

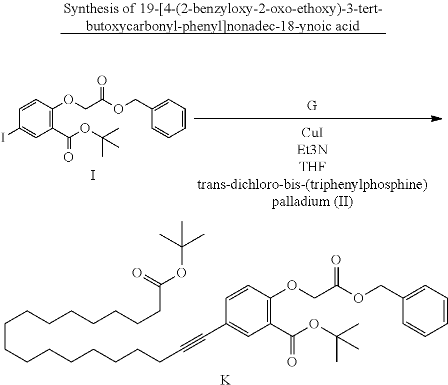

General Procedure: I (0.98 g, 2.1 mmol) and triethylamine (2 mL) were added to a suspension of trans-dichloro-bis(triphenylphosphine)palladium (0.03 g, 0.043 mmol) and copper(I)iodide (0.017 g, 0.086 mmol) in THF (10 mL), and then G (0.8 g, 23 mmol) was added to the mixture and stirred at room temperature for 24 h. The mixture was concentrated in vacuo and filtered through a plug of Celite and silica with diethyl ether (3×25 mL). The organic solution was concentrated in vacuo and purified by flash chromatography (10% dichloromethane in petroleum ether 40-60) to give the product K. (1.3 g, 90%)

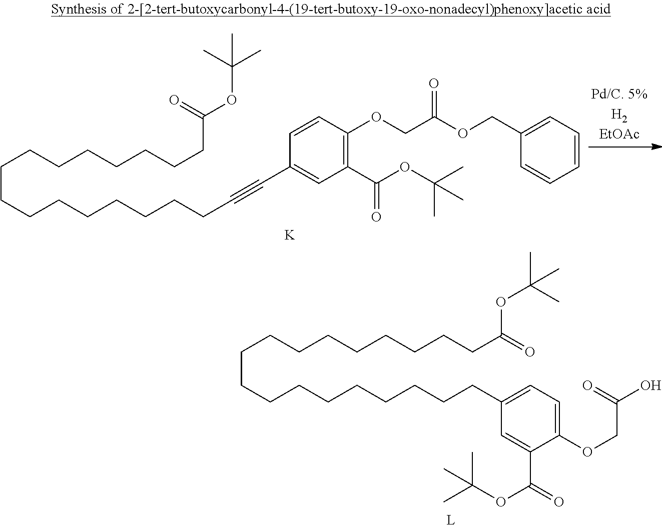

General Procedure: A mixture of K (1.2 g, 1.73 mmol) and Pd/C (0.1 g) in ethyl acetate (10 mL) was stirred under hydrogen atmosphere for 16 hours. The mixture was filtered and concentrated under reduced pressure to obtain L (1 g, 1.65 mmol).

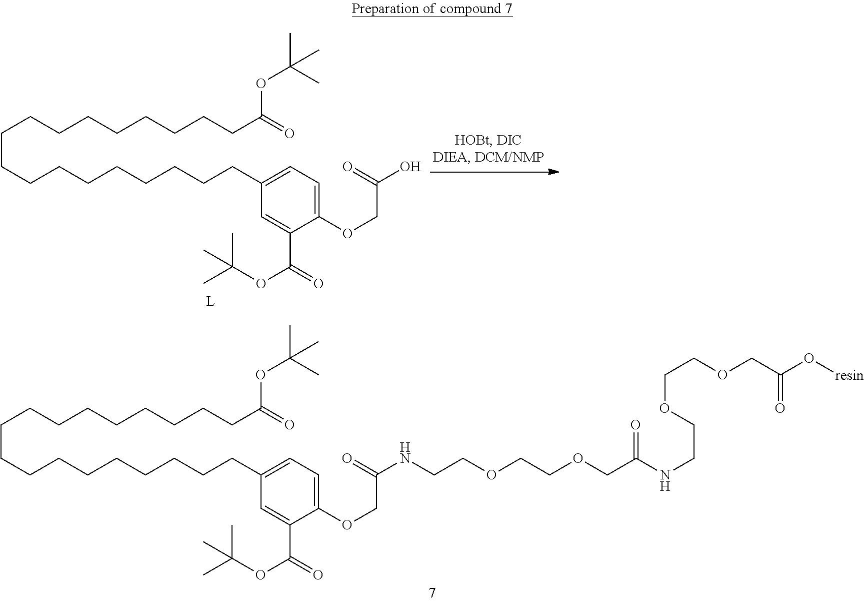

General Procedure: To a solution of compound L (1 g, 1.65 mmol) in NMP (5 mL) and dichloromethane (5 mL) was added HOBt (0.243 g, 1.8 mmol) and diisopropylcarbodiimide (0.227 g, 1.8 mmol). The mixture was stirred at 5-10° C. for 1 h, then added to compound 6 (0.825 g, crude, 0.55 mmol in theory), the DIEA (0.232 g, 1.8 mmol) was added. The mixture was vortexed at 5-10° C. for 16 hrs. The mixture was filtered, washed with NMP (3×10 mL), DCM (3×10 mL) and NMP (3×10 mL) to give compound 7 (1.3 g, crude) as yellow solid, which was used in next step directly. A positive TNBS test gave colorless resins

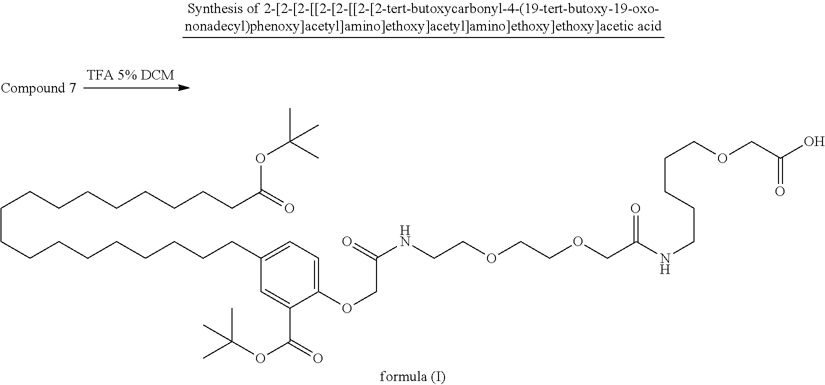 [SMILES: CC(C)(C)OC(=O)CCCCCCCCCCCCCCCCCCc1ccc(OCC(=O)NCCOCCOCC(=O)NCCCCCOCC(=O)O)c(C(=O)OC(C)(C)C)c1]

General Procedure: 5% TFA/DCM (10 mL) was added to compound 7 (1.3 g, crude, 0.55 mmol in theory). The mixture was vortexed at 5-10° C. for 20 min, filtered, the residue was treated with the same procedure twice. The residue was washed with DCM (3×60 mL). The combined organic phase was cooled to 0° C., diluted with water (200 mL), neutralized with saturated sodium bicarbonate solution to pH=3, the organic phase was separated, the aqueous phase was extracted with DCM (3×100 mL). The organic phase was dried over sodium sulfate, filtered and concentrated, the residue was purified pre-HPLC (TFA) to give Formula (I) (0.180 g, yield: 38%) as white solid.

### Synthesis Section 2: Conjugate

Provided herein are insulin conjugates comprising inter alia a human serum albumin binder of Formula (I):

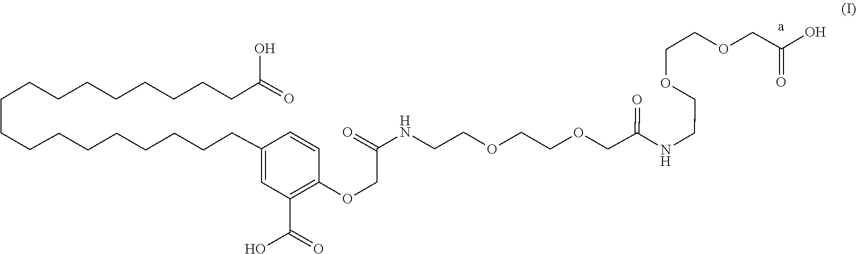 [SMILES: O=C(O)CCCCCCCCCCCCCCCCCCc1ccc(OCC(=O)NCCOCCOCC(=O)NCCOCCOCC(=O)O)c(C(=O)O)c1]

The human serum albumin binder of Formula (I) is covalently bound to the insulin conjugate in that the terminal carboxy group “a” of the compound of Formula (I) is covalently bound to the epsilon amino group of lysine B29.

Pharmaceutically acceptable salts of the insulin conjugates include acid addition and base salts. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinafoate salts. Suitable base salts are formed from bases which form non-toxic salts. Examples include the aluminium, arginine, benzathine, calcium, choline, diethylamine, bis(2-hydroxyethyl)amine (diolamine), glycine, lysine, magnesium, meglumine, 2-aminoethanol (olamine), potassium, sodium, 2-amino-2-(hydroxymethyl)propane-1,3-diol (tris or tromethamine) and zinc salts. Hemisalts of acids and bases may also be formed, for example, hemisulfate and hemicalcium salts. For a review on suitable salts, see Handbook of Pharmaceutical Salts: Properties, Selection, and Use by Stahl and Wermuth (Wiley-VCH, 2002).

The conjugates, and pharmaceutically acceptable salts thereof, may exist in unsolvated and solvated forms. The term ‘solvate’ is used herein to describe a molecular complex comprising the compound of Formula I, or a pharmaceutically acceptable salt thereof, and one or more pharmaceutically acceptable solvent molecules, for example, ethanol. The term ‘hydrate’ is employed when said solvent is water.

Examples of isotopes suitable for inclusion in the conjugates include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulfur, such as ³⁵S.

Certain isotopically-labelled conjugates, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled conjugates can generally be prepared by conventional techniques known to those skilled in the art.

Pharmaceutically acceptable solvates in accordance with the invention include those wherein the solvent of crystallization may be isotopically substituted, e.g. D₂O, d₆-acetone, d₆-DMSO.

In some embodiments, the insulin conjugate is an insulin conjugate as per the first aspect above. The definitions and explanations provided above apply accordingly.

### Synthesis Section 3: Human Insulin Analog and Conjugate Synthesis

### 3.1 Human Insulin

The amino acid sequences of the A and B chain of human insulin are:
A-chain: GIVEQCCTSICSLYQLENYCN (SEQ ID NO: 1)
B-chain: FVNQHLCGSHLVEALYLVCGERGFFYTPKT (SEQ ID NO: 2)

An intrachenar disulfide bridge is present between Cys(A6) and Cys(A11), two interchenar disulfide bridges are present between Cys(A7) and Cys(B7) and between Cys(A20) and Cys(B19).

### 3.2 Conjugate 1

Conjugate 1 is based on human insulin with mutations in positions A5, A14, A18, A21, B16, B25, B27 and an addition of the amino acid at position B31:
Arg(A5): The amino acid at position 5 of the A-chain of human insulin (Q, glutamine, Gin) is substituted by arginine (R, Arg),
Glu(A14): The amino acid at position 14 of the A-chain of human insulin (Y, tyrosine, Tyr) is substituted by glutamic Acid (E, Glu),
Arg(A18): The amino acid at position 18 of the A-chain of human insulin (N, asparagine, Asn) is substituted by arginine (R, Arg),
Gly(A21): The amino acid at position 21 of the A-chain of human insulin (N, asparagine, Asn) is substituted by arginine (G, Gly),
Glu(B16): The amino acid at position 16 of the B-chain of human insulin (Y, tyrosine, Tyr) is substituted by glutamic acid (E, Glu),
His(B25): The amino acid at position 25 of the B-chain of human insulin (F, phenylalanine, Phe) is substituted by histidine (H, His),
Arg(B27): The amino acid at position 25 of the B-chain of human insulin (T, threonine, Thr) is substituted by Arg (R, Arg),
Arg(B31): The amino acid arginine at position 31 of the B-chain of human insulin is added.

The complete amino acid sequence of Conjugate 1 in view of A and B chain is:
A-chain: GIVERCCTSICSLEQLERYCG (SEQ ID NO: 13)
B-chain: FVNQHLCGSHLVEALELVCGERGFHYRPKTR (SEQ ID NO: 9)

The one intrachenar and the two interchenar disulfide bridges are in accordance with human insulin.

3.3 Conjugate 1 with Insulin Analog/Synthesis of Arg(A5)Glu(A14)Arg(A18)Gly(A21)Glu(B16)His(B25)Arg(B27)2-[2-[2-[2-[2-[2-[2-(carbonylmethoxy)ethoxy]ethylamino]-2-oxo-ethoxy]ethoxy]ethylamino]-2-oxo-ethoxy]-5-(18-carboxyoctadecyl)benzoic acid Lys(B29)Arg(B31)-insulin.

A conjugate was prepared from Arg(A5)Glu(A14)Arg(A18)Gly(A21)Glu(B16)His(B25)Arg(B27)Arg(B31)-insulin according to 3.2 and tert-butyl 5-(19-tert-butoxy-19-oxo-nonadecyl)-2-[2-[2-[2-[2-[2-[2-[2-(2,5-dioxopyrrolidin-1-yl)oxy-2-oxo-ethoxy]ethoxy]ethylamino]-2-oxo-ethoxy]ethoxy]ethylamino]-2-oxo-ethoxy]benzoate:

### Synthesis of tert-butyl 5-(19-tert-butoxy-19-oxo-nonadecyl)-2-[2-[2-[2-[2-[2-[2-[2-(2,5-dioxopyrrolidin-1-yl)oxy-2-oxo-ethoxy]ethoxy]ethylamino]-2-oxo-ethoxy]ethoxy]ethylamino]-2-oxo-ethoxy]benzoate

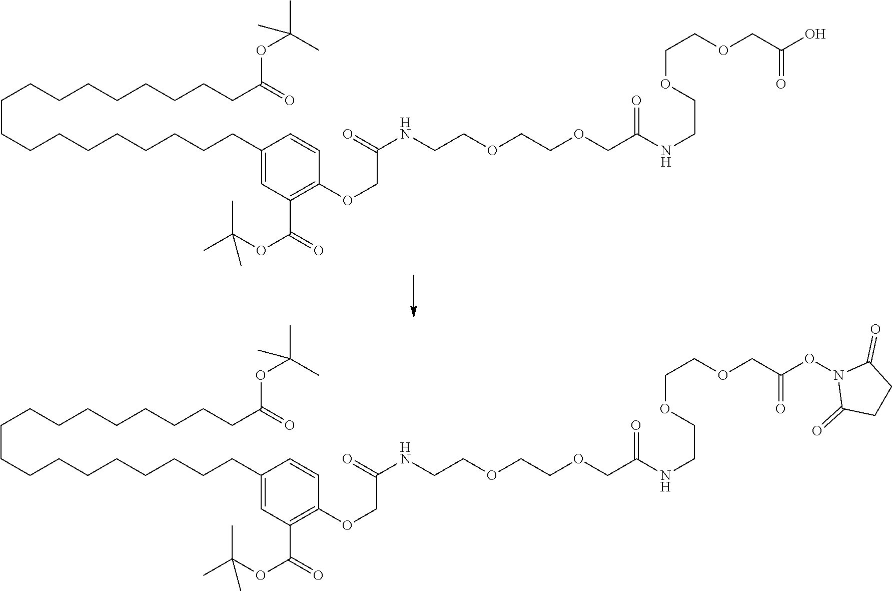 [SMILES: CC(C)(C)OC(=O)CCCCCCCCCCCCCCCCCCc1ccc(OCC(=O)NCCOCCOCC(=O)NCCOCCOCC(=O)O)c(C(=O)OC(C)(C)C)c1 ; CC(C)(C)OC(=O)CCCCCCCCCCCCCCCCCCc1ccc(OCC(=O)NCCOCCOCC(=O)NCCOCCOCC(=O)ON2C(=O)CCC2=O)c(C(=O)OC(C)(C)C)c1]

To a solution of 29 mg 2-[2-[2-[2-[2-[2-[[2-[2-tert-butoxycarbonyl-4-(19-tert-butoxy-19-oxo-nonadecyl)phenoxy]acetyl]amino]ethoxy]ethoxy-acetyl]amino]ethoxy]ethoxy]acetic acid in 9 ml DMF, 92.7 μl triethylamine, 106 mg TSTU and a trace of DMAP were added. The solution was stirred for one hour. The product was used without further purification. The conversion to the O-succinimide ester was 65%.

A solution of 500 mg insulin analog was suspended in 25 ml water and then 0.45 ml triethylamine was added. To the clear solution 25 ml MeCN and then 0.9 ml (45.89 mM in DMF) tert-butyl 5-(19-tert-butoxy-19-oxo-nonadecyl)-2-[2-[2-[2-[2-[2-[2-[2-(2,5-dioxopyrrolidin-1-yl)oxy-2-oxo-ethoxy]ethoxy]ethylamino]-2-oxo-ethoxy]ethoxy]ethylamino]-2-oxo-ethoxy]benzoate were added. The solution was stirred for 1 hour at room temperature. The reaction was analyzed with waters UPLC H-class at 214 nm in a sodium chloride phosphate buffer. Waters BEH300 10 cm. The product was purified by HPLC with Äcta avant 25.
Kinetex 5 μm C18 100 A 250×21.2 mm. Column volume (CV) 88 ml.
Column volume (CV) 88 ml.
Solvent A: 0.5% acetic acid in water
Solvent B: 0.5 acetic acid in water/MeCN 2:8
Gradient 95% A 5% B to 40% A60% B in 14 CV

The reaction was analyzed with waters UPLC H-class at 214 nm in a sodium chloride phosphate buffer. Waters BEH300 10 cm. The solution was lyophilized and gave the desired product. 98 mg 34% yield. Mass spec.: 6859 g/mol.

After lyophilisation of the product, the powder was dissolved in 2 ml trifluor acetic acid. After one hour, the solution was neutralized with diluted sodium bicarbonate to pH 4. The product was purified by HPLC with Acta avant 25. Kinetex 5 μm C18 100 A 250×21.2 mm. Column volume (CV) 88 ml.
Solvent A: 0.5% acetic acid in water
Solvent B: 0.5% acetic acid in water/MeCN 4:6
Gradient 70% A 30% B to 30% A70% B in 8 CV

The reaction was analyzed with waters UPLC H-class at 214 nm in a sodium chloride phosphate buffer.
Waters BEH300 10 cm.

The solution was lyophilized and gave the desired product.
60 mg 62% yield.
Mass spec.: 6746.8 g/mol.

### 4. Analytical Data

### 4.1 Liquid Chromatography Mass Spectrometry (LCMS) Analysis

| Mass | | Mass |
|---|---|---|
| spectrometry | | spectrometry |
| Method | Method description | type |
| a | Waters SQD Single Quadrupol, 0.13 s | UPLC esi |
| | scantime for mass 100-1400 | |
| b | Waters SQD Single Quadrupol, 0.5 s | UPLC esi |
| | scantime for mass 100-1400 | |

| Liquid chromatography | |
|---|---|
| Method | Method description |
| A | Waters ACQUITY UPLC BEH C18 1.7 um 2.1 × 50 mm MV Kit; |
| | H2O + 0.05% TFA:MeCN + 0.035% TFA 98:2(0 min) to |
| | 98:2(0.2 min) to 2:98(3.8 min) to 2:98(4.3 min) to 98:2(4.5 min), 1 |
| | ml/min 55° C. |
| B | Waters ACQUITY UPLC BEH C18 1.7 um 2.1 × 50 mm; |
| | H2O + 0.05% TFA:MeCN + 0.035% TFA 95:5(0 min) to 5:95(2 min) |
| | to 5:95(2.6 min) to 95:5(2.7 min) to 95:5(3 min), 0.9 ml/min 55° C. |
| C | Waters ACQUITY UPLC BEH C18 1.7 um 2.1 × 50 mm; |
| | H2O + 0.05% TFA:MeCN + 0.035% TFA 98:2(0 min) to |
| | 98:2(0.2 min) to 2:98(3.8 min) to 2:98(4.3 min) to 98:2(4.5 min), 1 |
| | ml/min 55° C. |

### 5. Insulin Receptor Binding Affinity

Insulin receptor binding affinity for the insulin and Conjugate 1 was determined as described in Hartmann et al. (Effect of the long-acting insulin conjugates glargine and degludec on cardiomyocyte cell signaling and function. Cardiovasc Diabetol. 2016; 15:96). Isolation of insulin receptor embedded plasma membranes (M-IR) and competition binding experiments were performed as previously described (Sommerfeld et al., PLoS One. 2010; 5(3): e9540). Briefly, CHO-cells overexpressing the IR were collected and re-suspended in ice-cold 2.25 STM buffer (2.25 M sucrose, 5 mM Tris-HCl pH 7.4, 5 mM MgCl₂, complete protease inhibitor) and disrupted using a Dounce homogenizer followed by sonication. The homogenate was overlaid with 0.8 STM buffer (0.8 M sucrose, 5 mM Tris-HCl pH 7.4, 5 mM MgCl₂, complete protease inhibitor) and ultra-centrifuged for 90 min at 100,000 g. Plasma membranes at the interface were collected and washed twice with phosphate buffered saline (PBS). The final pellet was re-suspended in dilution buffer (50 mM Tris-HCl pH 7.4, 5 mM MgCl₂, complete protease inhibitor) and again homogenized with a Dounce homogenizer. Competition binding experiments were performed in a binding buffer (50 mM Tris-HCl, 150 mM NaCl, 0.1% BSA, complete protease inhibitor, adjusted to pH 7.8) in 96-well microplates. In each well 2 μg isolated membrane were incubated with 0.25 mg wheat germ agglutinin polyvinyltoluene polyethylenimine scintillation proximity assay (SPA) beads. Constant concentrations of [125I]-labelled human Ins (100 pM) and various concentrations of respective unlabelled Ins (0.001-1000 nM) were added for 12 h at room temperature (23° C.). The radioactivity was measured at equilibrium in a microplate scintillation counter (Wallac Microbeta, Freiburg, Germany).”

The insulin receptor binding affinity relative to human insulin for Conjugate 1 is depicted in Table 2.

TABLE 2
Insulin receptor B binding affinity relative to human insulin

| Human insulin | 100% |
|---|---|
| Conjugate 1 | <2% |

### Example 1: Production of Human Insulin and Insulin Conjugates

Human insulin as well as insulin conjugates were produced recombinantly. Polynucleotides encoding pre-pro-insulin were ordered from Geneart®. The designed polynucleotides were optimized for expression in yeast. They were inserted into an expression vector by classical restriction cloning enabling functional expression and secretion in Kluyveromyces lactis K. As secretion leader, the gene was C-terminally fused to a DNA sequence encoding the alpha mating factor signal of Saccharomyces cerevisiae. The recombinant gene expression was controlled by a lactose inducible K. lactis promoter.

Human insulin as well as insulin conjugates were manufactured as a pre-pro-insulin. A genetically fused N-terminal pre-sequence was used to improve expression and secretion yields and to stabilize the peptide in the culture broth. A broad variety of sequences can be used for this purpose and were tested for efficiency. The proinsulin itself consists of a B-chain fused to a C-peptide followed by the C-terminal A-chain. As C-peptide a variety of amino acid combinations are described. It was shown, that short peptides of 1-10 amino acids work well as C-sequences. For later processing of the insulin the recognition sites for specific proteases, which flank the C-peptide to enable its excision, are important.

K. lactis cells were made competent by chemical means. Subsequently, cells were transformed with the expression plasmid coding for the respective pre-pro-insulin. After insertion of the plasmid, cells were plated on selective agar plates containing geneticin. Grown colonies were isolated and tested for recombinant gene expression. Cells were grown to sufficiently high cell densities in yeast peptone dextrose medium supplemented with geneticin. After an initial growth phase, a salt-buffered yeast extract medium with geneticin supplemented with lactose was added to the cultures to induce expression of the recombinant gene. Cultures were grown several days and supernatants were harvested by centrifugation.

Purification of the functional insulin or insulin conjugates was started by a filtration procedure. Initial chromatographic capturing procedure was made with an ion-exchange resin. Cleavage of pre-pro-insulin to insulin was performed with the highly specific protease trypsin or endoproteinase Lys-C. Depletion of host cell protein, pre-sequence and product related products were made by a cascade of two additional chromatographic steps. Next to a hydrophobic interaction chromatography another ion-exchange procedure was applied to achieve this goal. Final polishing was made by reverse phase chromatography. Filtration, precipitation and freeze drying were used to finish the production process of the insulin molecule.

After coupling reactions with an activated carboxylic acid derivative, the solution with conjugated insulin molecules was filtered. Final purification was made by reverse phase chromatography. Filtration, precipitation and freeze drying were used to finish the synthesis of the target molecule.

Mutations of Conjugate 1 (human insulin analog moiety) in comparison with human insulin e.g. at positions B16, B25 and/or A14, were generated. Table 1 provides an overview of the generated insulins.

TABLE 1
Generated analogs of human insulin

| | | | | | | | | | | Amino acid se- | Amino acid se- |
|---|---|---|---|---|---|---|---|---|---|---|---|
| Analog | Backbone | A5 | A14 | A18 | A21 | B16 | B25 | B27 | B31 | quence Chain A | quence Chain B |
| WT | human | Gln | Tyr | Asn | Asn | Tyr | Phe | Thr | | GIVEQCCT- | FVNQHLCGS- |
| | Insulin | | | | | | | | | SICSLYQLE- | HLVEA- |
| | (wild-type) | | | | | | | | | NYCN | LYLVCGERG- |
| | | | | | | | | | | (SEQ ID NO: 1) | FFYTPKT |
| | | | | | | | | | | | (SEQ ID NO: 2) |
| 1 | Arg(A5)Glu | Arg | Glu | Arg | Gly | Glu | His | Arg | Arg | GIVERCCT- | FVNQHLCGS- |
| | (A14)Arg | | | | | | | | | SICSLEQLERY | HLVEALEL- |
| | (A18)Gly | | | | | | | | | CG | VCGERG- |
| | (A21)Glu | | | | | | | | | (SEQ ID NO: | FHYRPKTR |
| | (B16)His | | | | | | | | | 13) | (SEQ ID NO: 9) |
| | (B25)Arg | | | | | | | | | | |
| | (B27)Arg | | | | | | | | | | |
| | (B31)- | | | | | | | | | | |
| | Insulin | | | | | | | | | | |

### Example 2: Insulin Receptor Binding Affinity Assays/Insulin Receptor Autophosphorylation Assays

Insulin binding and signal transduction of various generated insulin conjugates were determined by a binding assay and a receptor autophosphorylation assay.

### A) Insulin Receptor Binding Affinity Assay

Insulin receptor binding affinity for the conjugates listed in Table 4 was determined as described in Hartmann et al. (Effect of the long-acting insulin conjugates glargine and degludec on cardiomyocyte cell signaling and function. Cardiovasc Diabetol. 2016; 15:96): Isolation of insulin receptor embedded plasma membranes (M-IR) and competition binding experiments were performed as previously described (Sommerfeld et al., PLoS One. 2010; 5(3): e9540). Briefly, CHO-cells overexpressing the IR were collected and re-suspended in ice-cold 2.25 STM buffer (2.25 M sucrose, 5 mM Tris-HCl pH 7.4, 5 mM MgCl2, complete protease inhibitor) and disrupted using a Dounce homogenizer followed by sonication. The homogenate was overlaid with 0.8 STM buffer (0.8 M sucrose, 5 mM Tris-HCl pH 7.4, 5 mM MgCl2, complete protease inhibitor) and ultra-centrifuged for 90 min at 100,000 g. Plasma membranes at the interface were collected and washed twice with phosphate buffered saline (PBS). The final pellet was re-suspended in dilution buffer (50 mM Tris-HCl pH 7.4, 5 mM MgCl2, complete protease inhibitor) and again homogenized with a Dounce homogenizer. Competition binding experiments were performed in a binding buffer (50 mM Tris-HCl, 150 mM NaCl, 0.1% BSA, complete protease inhibitor, adjusted to pH 7.8) in 96-well microplates. In each well 2 μg isolated membrane were incubated with 0.25 mg wheat germ agglutinin polyvinyltoluene polyethylenimine scintillation proximity assay (SPA) beads. Constant concentrations of [125I]-labelled human Ins (100 pM) and various concentrations of respective unlabelled Ins (0.001-1000 nM) were added for 12 h at room temperature (23° C.). The radioactivity was measured at equilibrium in a microplate scintillation counter (Wallac Microbeta, Freiburg, Germany).”

The results of the insulin receptor binding affinity assays for the tested conjugates relative to human insulin are shown in Table 5.

### B) Insulin Receptor Autophosphorylation Assays (as a Measure for Signal Transduction)

In order to determine signal transduction of an insulin conjugate binding to insulin receptor B, autophosphorylation was measured in vitro.

CHO cells expressing human insulin receptor isoform B (IR-B) were used for IR autophosphorylation assays using In-Cell Western technology as previously described (Sommerfeld et al., PLoS One. 2010; 5(3): e9540). For the analysis of IGF1R autophosphorylation, the receptor was overexpressed in a mouse embryo fibroblast 3T3 Tet off cell line (BD Bioscience, Heidelberg, Germany) that was stably transfected with IGF1R tetracycline-regulatable expression plasmid. In order to determine the receptor tyrosine phosphorylation level, cells were seeded into 96-well plates and grown for 44 h. Cells were serum starved with serum-free medium Ham's F12 medium (Life Technologies, Darmstadt, Germany) for 2 h. The cells were subsequently treated with increasing concentrations of either human insulin or the insulin conjugate for 20 min at 37° C. After incubation the medium was discarded and the cells fixed in 3.75% freshly prepared para-formaldehyde for 20 min. Cells were permeabilised with 0.1% Triton X-100 in PBS for 20 min. Blocking was performed with Odyssey blocking buffer (LICOR, Bad Homburg, Germany) for 1 hour at room temperature. Anti-pTyr 4G10 (Millipore, Schwalbach, Germany) was incubated for 2 h at room temperature. After incubation of the primary antibody, cells were washed with PBS+0.1% Tween 20 (Sigma-Aldrich, St Louis, MO, USA). The secondary antimouse-IgG-800-CW antibody (LICOR, Bad Homburg, Germany) was incubated for 1 h. Results were normalized by the quantification of DNA with TO-PRO3 dye (Invitrogen, Karlsruhe, Germany). Data were obtained as relative units (RU).

The results of the insulin receptor autophosphorylation assays for the tested insulin conjugate relative to human insulin are shown in Table 5.

TABLE 5
Relative insulin receptor binding affinities and autophosphorylation
activities (for the sequences, please see Table 4).

| | | Insulin- | |
|---|---|---|---|
| | | receptor | |
| | | binding | Autophosphorylation |
| | Insulin Backbone | affinity* | activity* |
| WT | Human insulin (wild-type) | 1 | 1 |
| Conjugate | Arg(A5)Glu(A14)Arg(A18)Gly(A21)Glu(B16) | 0.01 | 0.07 |
| 1 | His(B25)Arg(B27)Arg(B31) Insulin | | |

*relative to human insulin,
nd: not determined

### C) Conclusions

Conjugate 1 still shows sufficient receptor binding and autophosphorylation activity to elucidate a pharmacological effect.

What is claimed is:

1. An insulin conjugate comprising a human serum albumin binder of Formula (I) and a human insulin analog, wherein the human serum albumin binder of Formula (I) is covalently bound to the human insulin analog in that the terminal carboxy group “a” of the human serum albumin binder of Formula (I) is covalently bound via an amide bond to the epsilon amino group of lysine B29 of the human insulin analog.

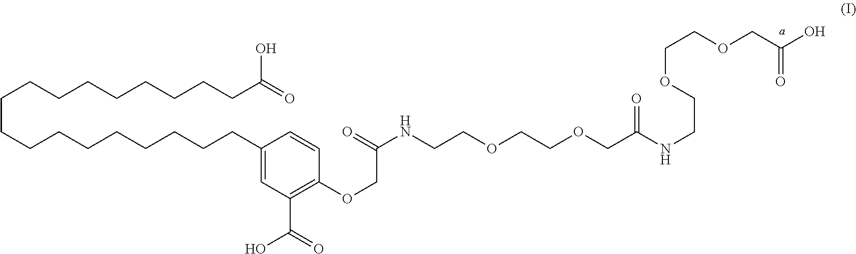 [SMILES: O=C(O)CCCCCCCCCCCCCCCCCCc1ccc(OCC(=O)NCCOCCOCC(=O)NCCOCCOCC(=O)O)c(C(=O)O)c1]

2. The insulin conjugate according to claim 1, comprising a human insulin analog having at least seven mutations relative to parent insulin, optionally wherein the mutations are selected from the group consisting of a substitution, deletion, and an addition of an amino acid residue.

3. The insulin conjugate according to any of the preceding claims, wherein the insulin conjugate comprises a human insulin analog having mutations:
at position A14 which is substituted with aspartic acid or glutamic acid, at position B16 which is substituted with histidine or glutamic acid,
at position B25 which is substituted with histidine,
and at least four additional substitutions,
and optionally the insulin conjugate has additional amino acid residues attached to the human insulin analog.

4. The insulin conjugate according to any of the preceding claims, wherein the insulin conjugate comprises a human insulin analog having mutations:
at position A14 which is substituted with aspartic acid or glutamic acid,
at position B16 which is substituted with glutamic acid,
at position B25 which is substituted with histidine,
and at least four additional substitutions,
optionally wherein all additional substitutions are to arginines, to glycines, or a combination thereof,
optionally wherein the insulin conjugate has additional amino acid residues attached to the human insulin analog.

5. The insulin conjugate according to any of the preceding claims, wherein the insulin conjugate comprises a human insulin analog having mutations
at position A14 which substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the insulin conjugate has additional amino acid residues attached to the human insulin analog.

6. The insulin conjugate according to any of the preceding claims, wherein the insulin conjugate comprises a human insulin analog having mutations:
at position A14 which is substituted with aspartic acid or glutamic acid,
at position B16 which is substituted with glutamic acid,
at position B25 which is substituted with histidine,
at position A21 which is substituted with glycine or arginine,
and at least three additional substitutions,
optionally wherein all additional substitutions are to arginines,
and optionally wherein the insulin conjugate has additional arginine residues attached to the human insulin analog.

7. The insulin conjugate according to any of the preceding claims, wherein the insulin conjugate comprises a human insulin analog selected from the group consisting of:
Arg(A5)Glu(A14)Arg(A15)Arg(A18)Arg(A21)Glu(B16)His(B25)Arg(B27)Arg(B31)insulin (human insulin);
Arg(A5)Glu(A14)Arg(A15)Arg(A18)Gly(A21)Glu(B16)His(B25)Arg(B27)Arg(B31)insulin (human insulin);
Arg(A5)Glu(A14)Arg(A18)Gly(A21)Glu(B16)His(B25)Arg(B27)Arg(B31)-insulin (human insulin);
Glu(A14)Arg(A15)Arg(A18)Arg(A21)Glu(B16)His(B25)Arg(B27)Arg(B31)-insulin (human insulin);
Arg(A5)Glu(A14)Arg(A18)Arg(A21)Glu(B16)His(B25)Arg(B27)Arg(B31)-insulin (human insulin);
Arg(A5)Glu(A14)Arg(A15)Arg(A21)Glu(B16)His(B25)Arg(B27)Arg(B31)-insulin (human insulin);
Arg(A5)Glu(A14)Arg(A15)Arg(A18)Arg(A21)Glu(B16)His(B25)Arg(B31)-insulin (human insulin).

8. The insulin conjugate according to any of the preceding claims, wherein the insulin conjugate comprises a human insulin analog selected from the group consisting of:
Arg(A5)Arg(A9)Glu(A14)Arg(A15)Arg(A18)Arg(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Arg(B3)Glu(B16)His(B25)Arg(B27)Des(B30)-insulin (human insulin);
Arg(A5)Arg(A9)Glu(A14)Arg(A15)Arg(A18)Arg(A21)Arg(B3)Glu(B16)His(B25)Des(B30)-insulin (human insulin);
Arg(A9)Glu(A14)Arg(A15)Arg(A18)Gly(A21)Arg(B3)Glu(B16)His(B25)Arg(B27)Des (B30)-insulin (human insulin);
Arg(A5)Glu(A14)Arg(A15)Arg(A18)Gly(A21)Arg(B3)Glu(B16)His(B25)Arg(B27)Des (B30)-insulin (human insulin);
Arg(A5)Arg(A9)Glu(A14)Arg(A18)Gly(A21)Arg(B3)Glu(B16)His(B25)Arg(B27)Des(B30)-insulin (human insulin);
Arg(A5)Arg(A9)Glu(A14)Arg(A15)Gly(A21)Arg(B3)Glu(B16)His(B25)Arg(B27)Des(B30)-insulin (human insulin);
Arg(A5)Arg(A9)Glu(A14)Arg(A15)Arg(A18)Gly(A21)Glu(B16)His(B25)Arg(B27)Des (B30)-insulin (human insulin);
Arg(A5)Arg(A9)Glu(A14)Arg(A15)Arg(A18)Gly(A21)Arg(B3)Glu(B16)His(B25)Des(B30)-insulin (human insulin);
Arg(A9)Glu(A14)Arg(A15)Arg(A18)Arg(A21)Arg(B3)Glu(B16)His(B25)Des(B30)-insulin (human insulin);
Arg(A5)Glu(A14)Arg(A15)Arg(A18)Arg(A21)Arg(B3)Glu(B16)His(B25)Des(B30)-insulin (human insulin);
Arg(A5)Arg(A9)Glu(A14)Arg(A18)Arg(A21)Arg(B3)Glu(B16)His(B25)Des(B30)-insulin (human insulin);
Arg(A5)Arg(A9)Glu(A14)Arg(A15)Arg(A18)Arg(A21)Glu(B16)His(B25)Des(B30)-insulin (human insulin);
Arg(A5)Arg(A9)Asp(A14)Arg(A15)Arg(A18)Arg(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Arg(B3)Glu(B16)His(B25)Arg(B27)Des(B30)-insulin (human insulin);
Arg(A5)Arg(A9)Asp(A14)Arg(A15)Arg(A18)Arg(A21)Arg(B3)Glu(B16)His(B25)Des(B30)-insulin (human insulin);
Arg(A9)Asp(A14)Arg(A15)Arg(A18)Gly(A21)Arg(B3)Glu(B16)His(B25)Arg(B27)Des (B30)-insulin (human insulin);
Arg(A5)Asp(A14)Arg(A15)Arg(A18)Gly(A21)Arg(B3)Glu(B16)His(B25)Arg(B27)Des (B30)-insulin (human insulin);
Arg(A5)Arg(A9)Asp(A14)Arg(A18)Gly(A21)Arg(B3)Glu(B16)His(B25)Arg(B27)Des(B30)-insulin (human insulin);
Arg(A5)Arg(A9)Asp(A14)Arg(A15)Gly(A21)Arg(B3)Glu(B16)His(B25)Arg(B27)Des(B30)-insulin (human insulin);
Arg(A5)Arg(A9)Asp(A14)Arg(A15)Arg(A18)Gly(A21)Glu(B16)His(B25)Arg(B27)Des (B30)-insulin (human insulin);
Arg(A5)Arg(A9)Asp(A14)Arg(A15)Arg(A18)Gly(A21)Arg(B3)Glu(B16)His(B25)Des(B30)-insulin (human insulin);
Arg(A9)Asp(A14)Arg(A15)Arg(A18)Arg(A21)Arg(B3)Glu(B16)His(B25)Des(B30)-insulin (human insulin);
Arg(A5)Asp(A14)Arg(A15)Arg(A18)Arg(A21)Arg(B3)Glu(B16)His(B25)Des(B30)-insulin (human insulin);
Arg(A5)Arg(A9)Asp(A14)Arg(A18)Arg(A21)Arg(B3)Glu(B16)His(B25)Des(B30)-insulin (human insulin);
Arg(A5)Arg(A9)Asp(A14)Arg(A15)Arg(A18)Arg(A21)Glu(B16)His(B25)Des(B30)-insulin (human insulin).

9. The insulin conjugate according to any of the preceding claims, wherein the insulin conjugate is Conjugate 1 (A chain sequence: SEQ ID NO: 13; B chain sequence: SEQ ID NO: 9): 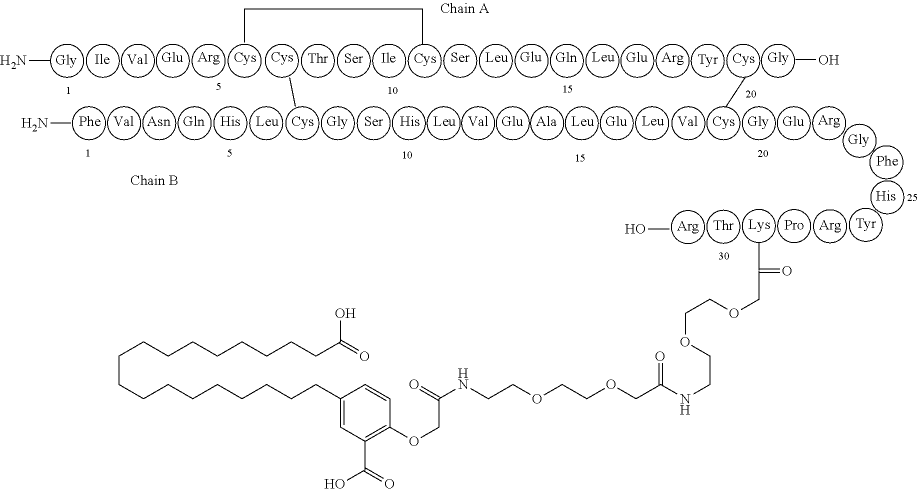
or
Conjugate 2 (A chain sequence: SEQ ID NO: 11; B chain sequence: SEQ ID NO: 9): 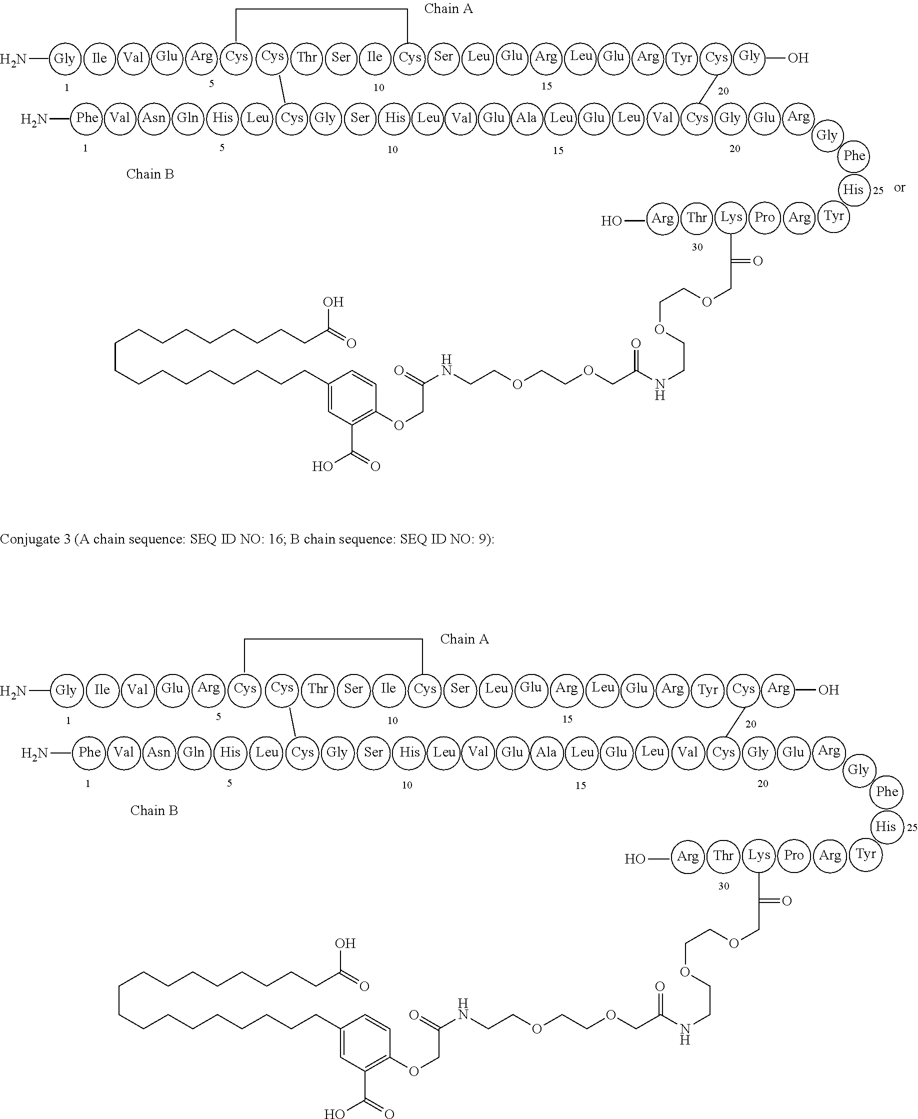
or
Conjugate 4 (A chain sequence: SEQ ID NO: 16; B chain sequence: SEQ ID NO: 8):

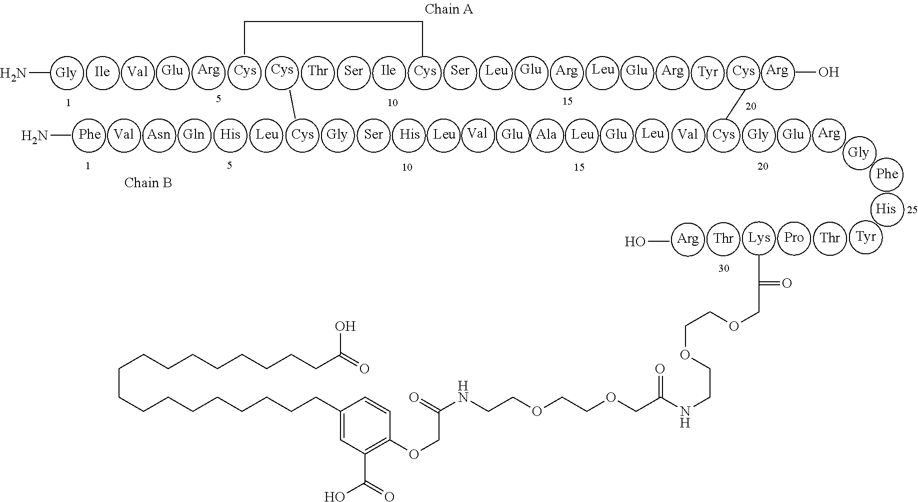

10. The insulin conjugate according to any of the preceding claims, wherein the insulin conjugate is Conjugate 5 (A chain sequence: SEQ ID NO: 17; B chain sequence: SEQ ID NO: 3): 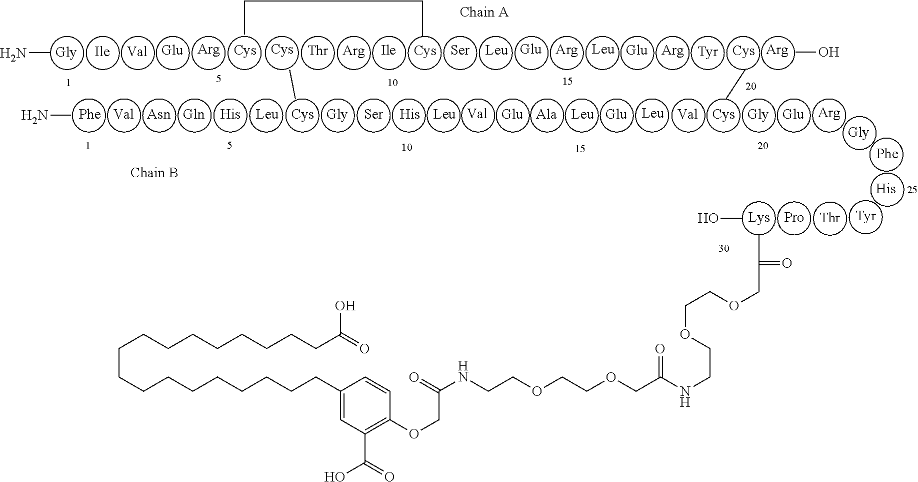
or
Conjugate 6 (A chain sequence: SEQ ID NO: 18; B chain sequence: SEQ ID NO: 3): 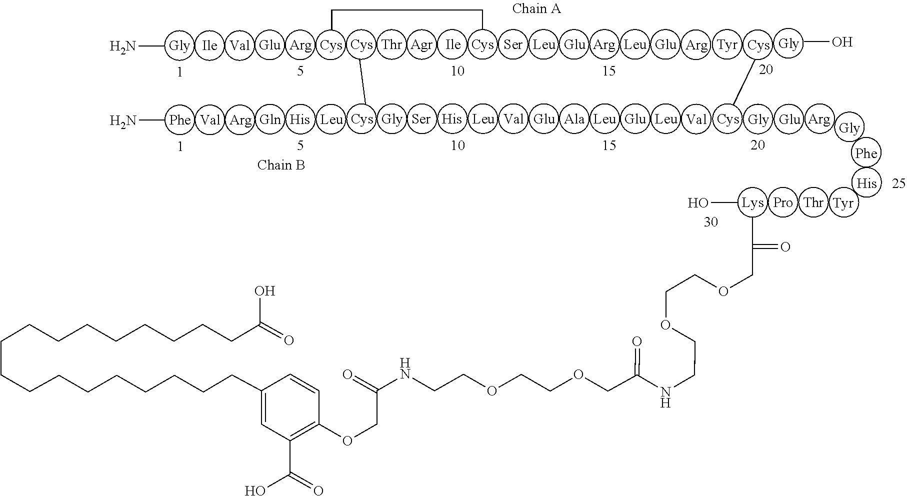
or
Conjugate 7 (A chain sequence: SEQ ID NO: 16; B chain sequence: SEQ ID NO: 3):

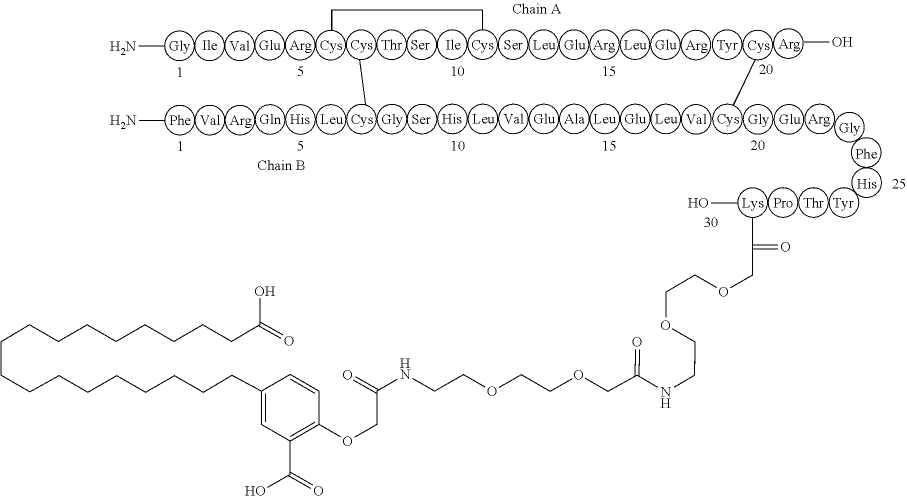

11. A human insulin analog as defined in any one of claims 3 to 10, such as a human insulin analog as defined in any of claims 7 to 10.

12. A pharmaceutical composition comprising in a pharmaceutically effective amount the insulin conjugate according to any of claims 1 to 10, or the insulin analog of claim 11.

13. An insulin conjugate according to any of claims 1 to 10, or the insulin analog of claim 11 for use as a medicament.

14. An insulin conjugate according to any of claims 1 to 10, or the insulin analog of claim 11 for use as a medicament for the treatment of a disease selected from the group consisting of gestational diabetes, diabetes mellitus type 1, diabetes mellitus type 2, and hyperglycemia and/or for lowering blood glucose levels.

15. An insulin conjugate according to any of claims 1 to 10, for use as a medicament for the treatment of diabetes mellitus type 2.
